(19) United States
(12) Patent Application Publication
(10) Pub. No.: US 2024/0309557 A1
(43) Pub. Date: Sep. 19, 2024

(54) PYRAZOLE AND PYRAZOLINE-CONTAINING PEPTIDES, HIGH THROUGHPUT CLICK LIBRARIES, AND METHODS

(71) Applicant: UNM Rainforest Innovations, Albuquerque, NM (US)

(72) Inventors: Brian Aaron Gold, Albuquerque, NM (US); Pavel Yamanushkin, Albuquerque, NM (US); Mark Aldren M. Feliciano, Albuquerque, NM (US)

(21) Appl. No.: 18/274,323

(22) PCT Filed: Jan. 26, 2022
(86) PCT No.: PCT/US2022/013895

§ 371 Date: Jul. 26, 2023

Related U.S. Application Data
(60) Provisional application No. 63/142,125, filed on Jan. 27, 2021.

(51) Int. Cl. C40B 50/08; C07K 1/06; C07K 7/06; C40B 40/10

(52) U.S. Cl. CPC C40B 50/08; C07K 1/062; C07K 1/063; C07K 7/06; C40B 40/10

(57) ABSTRACT

Pyrazole-containing peptides and pyrazoline containing peptides. The pyrazole-containing peptides and/or the pyrazoline containing peptides may include one or more cyclic peptides. Methods for making pyrazole-containing peptides and pyrazoline containing peptides. The method includes reacting a diazo functionalized amino acid with an alkyne or an alkene functionalized amino acid. Amino acids may be functionalized on the N-terminus and/or the C-terminus.

### CROSS-REFERENCE TO RELATED APPLICATION

This application claims the benefit of U.S. Provisional Patent Application No. 63/142,125, filed Jan. 27, 2021, which is incorporated herein by reference in its entirety.

### GOVERNMENT FUNDING

This invention was made with government support GM130422 awarded the National Institutes of Health. The government has certain rights in the invention.

### SUMMARY

This disclosure describes, in one aspect, a pyrazole-containing peptide of one of formulas,

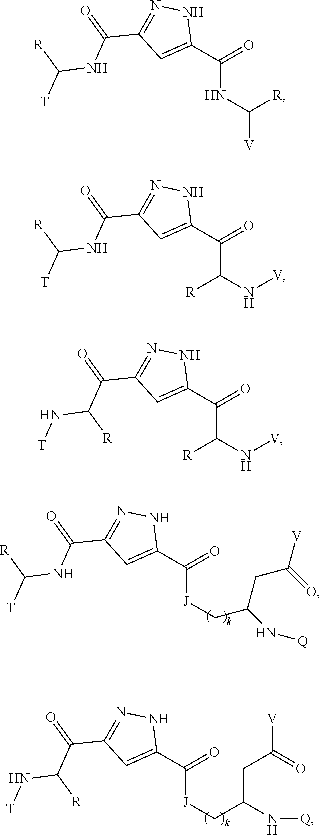

or an isomer thereof. Each R is independently an amino acid side chain. T, V, and Q are each independently an amino acid, a peptide, an amine, an OH, or a protecting group. Each k is 0, 1, 2, 3, 4, 5, 6, 7, or 8. J is one of the formulas

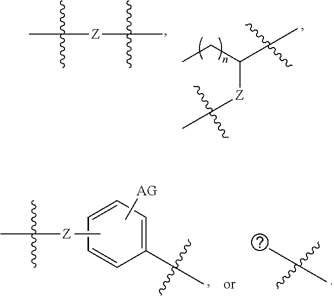

Each Z is independently O, N, NH, S, SH, or Se. Each n is 0, 1, 2, or 3. AG is a halogen or alkyl.

In another aspect, the present disclosure describes a pyrazoline-containing peptide of one of formulas,

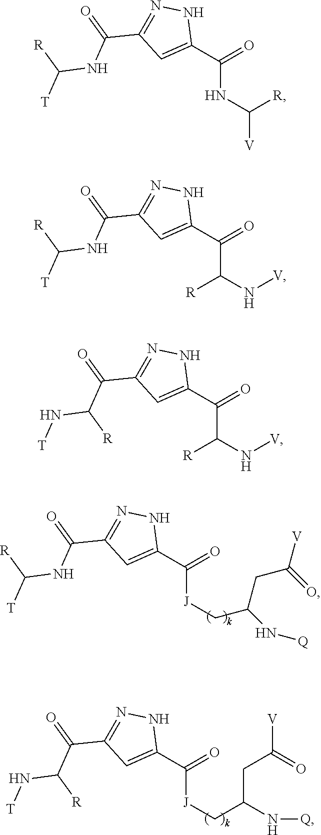

or an isomer thereof. Each R is independently an amino acid side chain. T, V, and Q are each independently an amino acid, a peptide, an amine, an OH, or a protecting group. Each k is 0, 1, 2, 3, 4, 5, 6, 7, or 8. J is one of the formulas

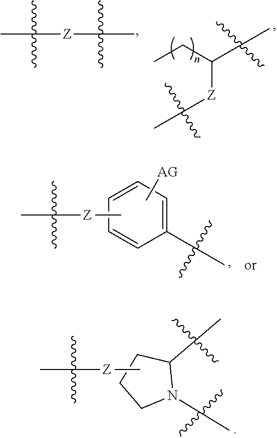

Each Z is independently O, N, NH, S, SH, or Se. Each n is 0, 1, 2, or 3. Each AG is a halogen or alkyl.

In some embodiments, T and V are peptides.

In some embodiments, T, V, and Q are peptides.

In some embodiments, T and Q are peptides.

In some embodiments, V and Q are peptides.

In some embodiments, T and V are covalently coupled through a cyclic peptide.

In some embodiments, V and Q are covalently coupled through a cyclic peptide.

In some embodiments, T is an amino acid and Vi

[symbol]

In some embodiments, T is an amino acid and Vi

[symbol]

In some embodiments, V is an amino acid and Tis H or C(O)OH.

[symbol]

In some embodiments, wherein T is an amino acid, V is an amino acid and Q is H or C(O)OH.

In some embodiments, T is an amino acid, Q is an amino acid and Vis H or C(O)OH.

In some embodiments, V is an amino acid, Q is an amino acid and T is H or C(O)OH.

In some embodiments, T is an amino acid, V is H or C(O)OH, and Q is H or C(O)OH.

In some embodiments, V is an amino acid, T is H or C(O)OH, and Q is H or C(O)OH.

In some embodiments, Q is an amino acid, T is H or C(O)OH, and Vis H or C(O)OH.

In some embodiments, each R is independently the side chain of a proteogenic amino acid.

In another aspect, the present disclosure describes a method for synthesizing a pyrazole-containing peptide or a pyrazoline-containing peptide. The method includes reacting an alkyne functionalized amino acid of formula

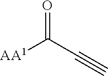

or a alkene functionalized amino acid of formula

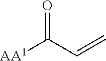

with a diazo functionalized amino acid of formula

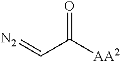

under a set of reaction conditions. AA¹ and AA² are each independently one of formulas:

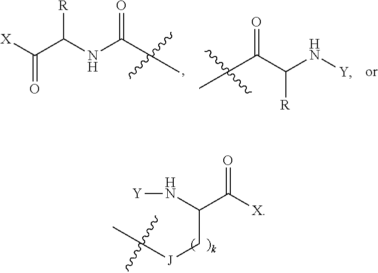

Each R is independently an amino acid side chain. X and Y are each independently C(O)OH, H, an amine protecting group or a carboxylic acid protecting group. Each k is 0, 1, 2, 3, or 4. J is of the of the general formula:

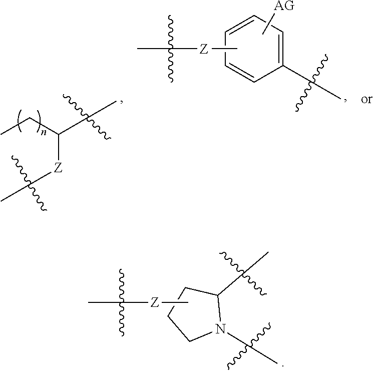

Each Z is independently O, N, NH, S, SH, or Se. Each n is 0, 1, 2, 3, or 4. AG is a halogen or alkyl.

In some embodiments, the the pyrazole-containin

[symbol]

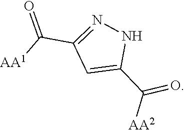

In some embodiments, the pyrazoline-containing peptide of formula

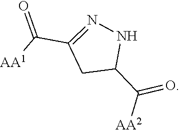

In some embodiments, AA¹ and AA² are

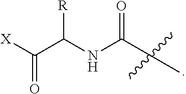

In some embodiments, AA¹ is

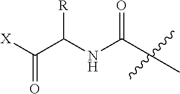

### and AA² is

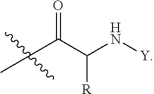

In some embodiments, AA¹ and AA² are

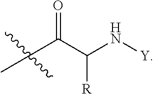

In some embodiments, AA¹ is

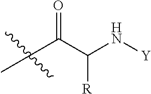

### and AA² is

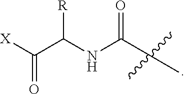

In some embodiments, AA¹ is

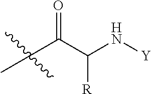

### and AA² is

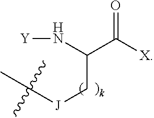

In some embodiments, AA¹ is

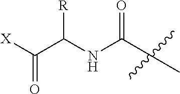

### and AA² is

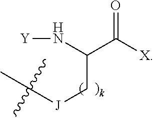

In some embodiments, AA¹ is

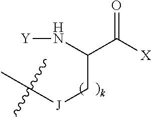

### and AA² is.

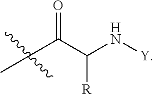

In some embodiments, AA¹ is

[symbol]

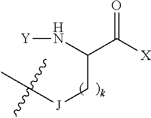

### and AA² is

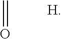

In some embodiments, AA¹ and AA² are

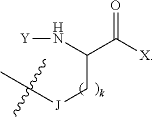

In some embodiments, each R is the side chain of a proteogenic amino acid.

In some embodiments, each R is the side chain of an unnatural amino acid.

In some embodiments, the reacting conditions comprise one or more of an aqueous environment, catalyst-free environment, ambient temperature, and ambient pressure.

In some embodiments, the method further includes removing one or more protecting groups.

In another aspect, the present disclosure describes a method of making a library of pyrazole-containing peptides comprising repeating the method any of the above-described aspects and/or embodiments to produce a plurality of pyrazole-containing peptides.

In another aspect, the present disclosure describes a method of making a library of pyrazoline-containing peptides comprising repeating the method any of the above-described aspects and/or embodiments to produce a plurality of pyrazoline-containing peptides.

In another aspect, the present disclosure describes a method a method of making a library of synthetic amino acid-derived compounds. The method includes providing building blocks comprising a set of diazo-functional amino acids and a set of alkene- or alkyne-functional amino acids. The method further includes reacting one of the set of diazo-functional amino acids with one of the set of alkene- or alkyne-functional amino acids under conditions effective to form a pyrazole- or pyrazoline-containing peptide cycloadduct of the library. The method further includes repeating the reacting step with a different diazo-functional amino acid and a different alkene- or alkyne-functional amino acid under conditions effective to form a different pyrazole- or pyrazoline-containing peptide cycloadduct of the library.

In some embodiments, the diazo-functional amino acid is an N-terminal diazo-functional amino acid (i.e., an N-terminal amino acid-based diazo compound or a diazoacetamide), a C-terminal diazo-functional amino acid (C-terminal amino acid-based diazo compound), or a side chain diazo-functional amino acid.

In some embodiments, the alkene- or alkyne-func [symbol] alkene- or alkyne-functional amino acid (i.e., an N-termin [symbol] compound), a C-terminal alkene- or alkyne-functional amino acid (C-terminal amino acid-based alkene or alkyne compound), or a side chain alkene or alkyne-functional amino acid.

In some embodiments, the building blocks include 60 N-terminal, 120 C-terminal, and 20 side-chain functionalized building blocks.

In some embodiments, the library comprises over 7,000 cycloadducts.

In some embodiments, the conditions comprise aqueous, catalyst-free 1,3-dipolar cycloaddition under ambient conditions (e.g., room temperature).

In some embodiments, the amino acid is Gly, Ala, Val, Ile, Leu, Met, Phe, Tyr, Trp, Pro, Cys, Lys, Ser, Thr, Asn, Gln, Arg, His, Asp, Glu, Hyp, Flp, Sec, Orn, or other common derivatives, and their enantiomers/diastereomers.

In some embodiments, the alkene- or alkyne-functional amino acids are reacted with trimethylsilyldiazomethane (TMS diazomethane).

In some embodiments, the pyrazole- or pyrazoline-containing cycloadduct is incorporated into larger cyclic or acyclic peptides.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 is a schematic representation of a 1,3-dipolar “click” cycloaddition reaction using an N-terminal diazo functionalized amino acid with a C-terminal alkyne functionalized amino acid to generate a pyrazole-containing peptide. R and R′ are each independently a side chain of an amino acid. R″ is a carboxylic acid protecting group and R′″ is an amine protecting group.

FIG. 2 shows exemplary N-terminal, C-terminal, and side chain functionalized alkyne, alkene, diazo compounds, and amino acids for generating pyrazole-containing peptide and pyrazoline-containing peptide libraries. Each R is a side chain of an amino acid.

### DETAILED DESCRIPTION OF ILLUSTRATIVE EMBODIMENTS

This disclosure provides pyrazole-containing pep [symbol] peptides that may be useful in drug discovery screening [symbol] development, and as drugs. Also described are methods of making libraries of pyrazole-containing peptides or pyrazoline-containing peptides using 1,3-dipolar cycloadditions “click chemistry.”

Human health relies on the perpetual discovery of novel therapeutics. Beginning with the isolation and derivatization of natural products, synthetic methods continue to transform the pharmaceutical sciences. Accelerated drug discovery is enabled by high-throughput screening, where libraries of synthetic compounds are rapidly assessed for biological activity. As the majority of FDA-approved drugs are small molecules, such approaches are common in the continued discovery of new drug compounds. While largely successful, overused synthetic methods narrow the explored chemical space and, thus, limit the structural diversity of drugs and drug fragments.

Approximately 75% of the pharmacophores used in fragment-based screens are linear or flat. The over-representation of two-dimensional pharmacophores is in stark contrast to biomolecules found in nature. The three-dimensional structure of proteins—and the relationship to function—is a central tenet of protein science, while the synthesis of complex natural products drove the “Golden Age” of organic chemistry. Among emerging areas addressing the lack of structural diversity in drug discovery are constrained-peptide therapeutics and organometallics drug fragments.

In one aspect, this disclosure describes pyrazole-containing peptides.

As used herein, the term “peptide” refers to two or more amino acids linked in a chain. Thus, amino acids of a “peptide,” as used herein, do not necessarily need to be linked by conventional amide peptide bonds. Instead, two amino acids may be linked in a chain through a heterocycle, such as a pyrazole or pyrazoline. Additionally, a “peptide,” as used herein, does not necessarily need to be linked in a chain such that there is an N-terminal amine or equivalent group and a C-terminal carboxylic acid or equivalent group. For example, a “peptide,” as used herein, may include an N-terminal amine or equivalent group and a C-terminal carboxylic acid or equivalent group; two N-terminal amines or equivalent groups; or two C-terminal carboxylic acids or equivalent groups.

In some embodiments, the pyrazole-containing peptide is of the general Formula I or an isomer thereof. In some embodiments, the pyrazole-containing peptide is of the general Formula II or an isomer thereof. In some embodiments, the pyrazole-containing peptide is of the general

Formula III or an isomer thereof. In some embodiments, [symbol] the general Formula IV or an isomer thereof. In some em [symbol] peptide is of the general Formula V or an isomer thereof.

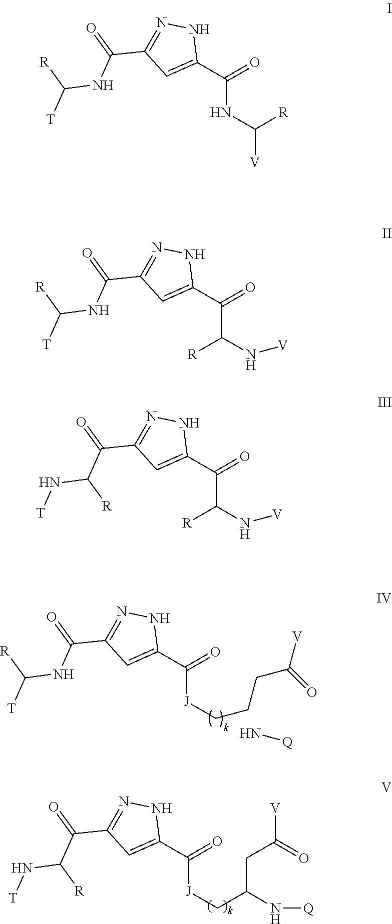

Generally, in each of Formulas I through V, each R is independently an amino acid side chain or a protected amino acid side chain. As used herein, a “protected amino acid side chain” refers to a reactive group on an amino acid side chain that is shielded from participating in a reaction through the use of protecting group. As used here, a “protecting group” refers to a temporary chemical group added to a reactive functional group to prevent the reactive functional group from reacting when the protecting group is present. Removal of the protecting reveals the reactive functional group. As used herein, a “reactive group” is a chemical group capable of participating in a reaction. Exemplary reactive groups on the side chains of amino acids include, but are not limited to, an alcohol, a thiol, an amine, a heterocycle, a guanidinium, or a carboxylic acid. Example protecting groups include but are not limited to tert-butyloxycarbonyl (Boc) fluorenylmethyloxycarbonyl (Fmoc), trityl (Trt), benzyloxycarbonyl (Cbz), allyloxycarbonyl (alloc), isobutene, 2,4-dimethoxybenzyl (Dub), benzyl (Bn), 2,2,4,6,7-pentamethyldihydrobenzofuran-5-sulfonyl (Pbf), 9-xanthyl (Xan), tosyl (Tos), benzyloxymethyl (Bom), formyl, allyl (Al), o-nitrobenzyl (ONB), p-methylbenzyl (Meb), or acetamidomethyl (Acm).

An artisan will appreciate that an amino acid side chain extends from the β carbon of an amino acid. In this disclosure, the carbon from which R extends is equivalent to the β carbon of the amino acid. In some embodiments, the amino acid side chain may be the side chain or protected side chain of any proteogenic amino acid. In some embodiments, the amino acid side chain may be side chain or protected side chain of any unnatural amino acid. In some embodiments, the amino acid side chain may be the side [symbol] Ala, Val, Ile, Leu, Met, Phe, Tyr, Trp, Pro, Cys, Lys, Ser

[symbol]

Hyp (hydroxyproline), Flp (fluoroproline), chloroproline, aminoproline, phosphoserine, phosphotyrosine, fluorotyrosine, fluorotryptophan, bromotryptophan, citrulline, norvaline, norleucine, N-methyl lysine (mono, di, or tri), N-methyl arginine (mono, di, or tri), 2,4-diaminobutyric acid, 2,3-diaminobutyric acid, penicillamine, Sec (selenocysteine), Orn (ornithine), or derivatives/analogues thereof. Any enantiomer or diastereomer of the amino acid side chain is contemplated. An artesian will appreciate that in embodiments where R is proline or a derivative/analog of proline, the nitrogen adjacent to the R group is a part of the proline ring. An artesian will also appreciate that in embodiments where R is proline or a derivative/analog of proline, the amine nitrogen adjacent to the R group does may not include a covalent bond to a H atom.

In some embodiments, the amino acid side chain is the side chain of Gly. In some embodiments, the amino acid side chain is the side chain of Ala. In some embodiments, the amino acid side chain is the side chain of Val. In some embodiments, the amino acid side chain is the side chain of Ile. In some embodiments, the amino acid side chain is the side chain of Leu. In some embodiments, the amino acid side chain is the side chain or protected side chain of Met. In some embodiments, the amino acid side chain is the side chain of Phe. In some embodiments, the amino acid side chain is the side or protected side chain of Tyr. In some embodiments, the amino acid side chain is the side chain or protected side chain of Trp. In some embodiments, the amino acid side chain is the side chain or protected side chain of Pro. In some embodiments, the amino acid side chain is the side chain or protected side chain of Cys. In some embodiments, the amino acid side chain is the side chain or protected side chain of Lys. In some embodiments, the amino acid side chain is the side chain or protected side chain of Ser. In some embodiments, the amino acid side chain is the side chain or protected side chain of Thr. In some embodiments, the amino acid side chain is the side chain or protected side chain of of Asn. In some embodiments, the amino acid side chain is the side chain or protected side chain of Gln. In some embodiments, the amino acid side chain is the side chain or protected side chain of Arg. In some embodiments, the amino acid side chain is the side chain or protected side chain of His. In some embodiments, the amino acid side chain is the side chain or protected side chain of Asp. In some embodiments, the amino acid side chain is the side chain or protected side chain of Glu. In some embodiments, the amino acid side chain is the side chain or protected side chain of Hyp. In some embodiments, the amino acid side chain is the side chain or protected side chain of Flp. In some embodiments, the amino acid side chain i [symbol] of Sec. In some embodiments, the amino acid side chain [symbol] of Orn. In some embodiments, the amino acid side chain is the side chain or protected side chain of chloroproline. In some embodiments, the amino acid side chain is the side chain or protected side chain of aminoproline. In some embodiments, the amino acid side chain is the side chain or protected side chain of phosphoserine. In some embodiments, the amino acid side chain is the side chain or protected side chain of phosphotyrosine. In some embodiments, the amino acid side chain is the side chain or protected side chain of fluorotyrosine. In some embodiments, the amino acid side chain is the side chain or protected side chain of fluorotryptophan. In some embodiments, the amino acid side chain is the side chain or protected side chain of bromotryptophan. In some embodiments, the amino acid side chain is the side chain or protected side chain of citrulline. In some embodiments, the amino acid side chain is the side chain or protected side chain of norvaline. In some embodiments, the amino acid side chain is the side chain or protected side chain of norleucine. In some embodiments, the amino acid side chain is the side chain or protected side chain of N-methyl lysine (mono, di, or tri). In some embodiments, the amino acid side chain is the side chain or protected side chain of N-methyl arginine (mono, di, or tri). In some embodiments, the amino acid side chain is the side chain, or protected side chain of, 2,4-diaminobutyric acid. In some embodiments, the amino acid side chain is the side chain, or protected side chain of, 2,3-diaminobutyric acid. In some embodiments, the amino acid side chain is the side chain or protected side chain penicillamine.

In Formula IV and V, J is a functionalized amino acid side chain. The amino acid side chain is functionalized such that it is covalently bonded to a terminal alkyne moiety or a terminal diazo moiety. In some embodiments J is of Formula J(I), (JII), J(III), or J(IV):

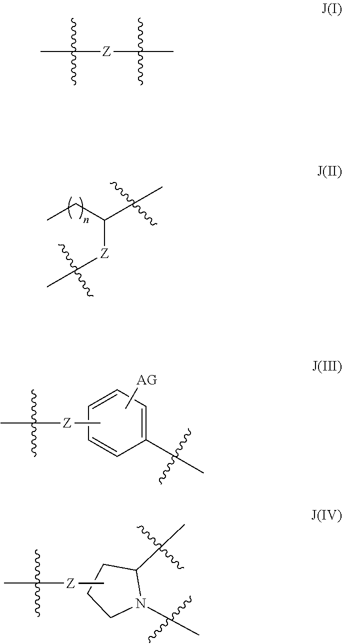

In Formulas J(I), J(II), J(III), and J(IV), Z is cova [symbol] moiety or a terminal diazo moiety. In Formulas of J(I), J( [symbol] oxygen (O), sulfur (S), selenium (Se) or a protonated or alkylated equivalent. In some embodiments, Z is N. In some embodiments, Z is NH. In some embodiments, Z is NH₂. In some embodiments, Z is O. In some embodiments, Z is S. In some embodiments, Z is SH. In some embodiments, Z is Se.

In the Formula J(II), n is 0, 1, 2, 3, 4, 5, 6, 7, or 8. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8.

In the Formula J(III), AG is a functional group such as a halogen, a hydroxyl, or an alkyl. In some embodiments AG is a halogen. In some embodiments, AG is a hydroxyl. In some embodiments, AG is an alkyl.

In the general Formulas IV and V, k is 0, 1, 2, 3, 4, 5, 6, 7, or 8. In some embodiments, k is 0. In some embodiments, k is 1. In some embodiments, k is 2. In some embodiments, k is 3. In some embodiments, k is 4. In some embodiments, k is 5. In some embodiments, k is 6. In some embodiments, k is 7. In some embodiments, k is 8.

In some embodiments when J is J(I) and Z is S, k is 1. In some embodiments, when J is J(I) and Z is O, k is 1.

In some embodiments when J is J(II), Z is O, n is 1, and k is 1. In some embodiments when J is J(I), k is 1 and Z is O. In some embodiments when J is J(I), k is 1 and Z is S.

In some embodiments when J is J(II), k is 1, n is 1 and Z is S. In some embodiments when J is J(II), k is 2, n is 1 and Z is S. In some embodiments when J is J(II), k is 1, n is 1 and Z is Se. In some embodiments when J is J(II), k is 2, n is 1 and Z is Se. In some embodiments when J is J(II), k is 1, n is 1 and Z is O. In some embodiments when J is J(II), k is 2, n is 1 and Z is O.

In some embodiments when J is J(III), k is 1 and Z is O. In some embodiments when J is J(III), k is 1 and Z is NH. In some embodiments when J is J(III), k is 1 and Z is NH₂. In some embodiments when J is J(III), k is 1 and Z is S. In some embodiments when J is J(III), k is 1 and Z is SH.

An artesian will appreciate that in Formula J(IV), k is 0 and the nitrogen in the five membered ring is the nitrogen that is covalently attached to Q in Formula IV or Formula V.

In the general Formulas IV through V, each of T, [symbol] acid, a peptide, a carboxylic acid (C(O)OH), H, or a prot [symbol] Q is an amino acid, a peptide, a carboxylic acid (C(O)OH), H, or a protecting group independent of the substituent present at the other locations.

In some embodiments, T is an amino acid or a peptide. In some embodiments when Tis an amino acid, the amino acid is an N-terminal amino acid. In some embodiments when T is a peptide, the peptide extends in the N-terminal direction such that the last amino acid in the peptide is an N-terminal amino acid. An artisan will recognize that when Tis covalently bonded to the carbon from which R extends, the amino acid will be an N-terminal amino acid. An artisan will recognize that when T is covalently bonded to the carbon from which R extends, the peptide will extend in the N-terminal direction such that the last amino acid in the peptide is an N-terminal amino acid. In some embodiments when T is an amino acid, the amino acid is a C-terminal amino acid. In some embodiments when T is a peptide, the peptide extends in the C-terminal direction such that the last amino acid in the peptide is a C-terminal amino acid. An artisan will recognize that when T is covalently to bonded to an amine adjacent from the carbon from which R extends, the amino acid will be a C-terminal amino acid. An artisan will recognize that when T is covalently to bonded to an amine adjacent from the carbon from which R extends, the peptide will extend in the C-terminal direction such that the last amino acid in the peptide is a C-terminal amino acid.

In some embodiments, V is an amino acid or a peptide. In some embodiments when V is an amino acid, the amino acid is a N-terminal amino acid. In some embodiments when Vis a peptide, the peptide extends in the N-terminal direction such that the last amino acid in the peptide is a N-terminal amino acid. An artisan will recognize that when Vis covalently bonded to the carbon from which R extends, the amino acid will be a N-terminal amino acid. An artisan will recognize that when Vis covalently bonded to the carbon from which R extends, the peptide will extend in the N-terminal direction such that the last amino acid in the peptide is a N-terminal amino acid. In some embodiments when V is an amino acid, the amino acid is a C-terminal amino acid. In some embodiments when V is a peptide, the peptide extends in the C-terminal direction such that the last amino acid in the peptide is a C-terminal amino acid. An artisan will recognize that when V is covalently to bonded to an amine adjacent from the carbon from which R extends, the amino acid will be a C-terminal amino acid. An artisan will recognize that when V is covalently to bonded to an amine adjacent from the carbon from which R extends, the peptide will extend in the C-terminal direc [symbol] peptide is a C-terminal amino acid.

In some embodiments, Q is an amino acid or a peptide. In some embodiments when Q is an amino acid, the amino acid is a N-terminal amino acid. In some embodiments when Q is a peptide, the peptide extends in the N-terminal direction such that the last amino acid in the peptide is a N-terminal amino acid. An artisan will recognize that when Q is covalently bonded to the carbon from which R extends, the amino acid will be a N-terminal amino acid. An artisan will recognize that when Q is covalently bonded to the carbon from which R extends, the peptide will extend in the N-terminus direction such that the last amino acid in the peptide is a N-terminal amino acid. In some embodiments where Q is an amino acid, the amino acid is a C-terminal amino acid. In some embodiments where Q is a peptide, the peptide extends in the C-terminal direction such that the last amino acid in the peptide is a C-terminal amino acid. An artisan will recognize that when Q is covalently to bonded to an amine adjacent from the carbon from which R extends, the amino acid will be a C-terminal amino acid. An artisan will recognize that when Q is covalently to bonded to an amine adjacent from the carbon from which Q extends, the peptide will extend in the C-terminal direction such that the last amino acid in the peptide is a C-terminal amino acid.

In some embodiments, a pyrazole-containing peptide may have two C-terminal amino acids. For example, in some embodiments, such as in Formula I, both T and V may be C-terminal amino acids or peptides that extend in the C-terminal direction such that the last amino acid in each peptide is a C-terminal amino acid. In some embodiments, such as in Formula IV and Formula V, both T and V can be C-terminal amino acids or peptides that extend in the C-terminal direction such that the last amino acid of each peptide is a C-terminal amino acid.

In some embodiments, a pyrazole-containing peptide may have two N-terminal amino acids. For example, in some embodiments, such as in Formula III, both T and V can be N-terminal amino acids or peptides that extend in the N-terminal direction such that the last amino acid in each peptide is an N-terminal amino acid. In some embodiments, such as in Formula IV and Formula V, both T and Q can be N-terminal amino acids or peptides that extend in the N-terminal direction such that the last amino acid of each peptide is an N-terminal amino acid.

In some embodiments, a pyrazole-containing peptide may have one C-terminal amino acid and one N-terminal amino acid. For example, in some embodiments, such as in Formula II, V may be a N-terminal amino acid or a peptide that extends in the N-terminal direction such that the last amino acid is a N-terminal amino acid and T may be a C-terminal amino acid or a peptide that extends in the C-terminal direction such that [symbol] amino acid.

In some embodiments, a pyrazole-containing peptide may have two C-terminal amino acids and one N-terminal amino acid. For example, in some embodiments, such as in Formula IV and V, Q may be a N-terminal amino acid or a peptide that extends in the N-terminal direction such that the last amino acid is a N-terminal amino acid and both T and V can be C-terminal amino acids or peptides that extend in the C-terminal direction such that the last amino acid is a C-terminal amino acid.

In some embodiments, a pyrazole-containing peptide may have one C-terminal amino acid and two N-terminal amino acids. For example, in some embodiments, such as in Formula VI, and V, Q and T may each be a N-terminal amino acid or a peptide that extends in the N-terminal direction such that the last amino acid is a N-terminal amino acid and V may be a C-terminal amino acid or a peptide that extends in the C-terminal direction such that the last amino acid is a C-terminal amino acid.

In some embodiments, T is a protecting group. In some embodiments, T is a carboxylic acid protecting group. Exemplary carboxylic acid protecting groups include, but are not limited to, a methyl ester, a t-butyl ester, a benzyl ester, or a S-t-butyl ester. In the context of Formulas I through VII, an artisan will appreciate the carbonyl covalently attached to the T group is included in the ester carboxylic acid protecting group. In some embodiments, T is an amine protecting group. Exemplary amine protecting groups include, but are not limited to, fluorenylmethyloxycarbonyl (Fmoc) or tert-butyloxycarbonyl (Boc).

In some embodiments, V is a protecting group. In some embodiments, V is a carboxylic acid protecting group. Exemplary carboxylic acid protecting groups include, but are not limited to, a methyl ester, a t-butyl ester, a benzyl ester, or a S-t-butyl ester. In the context of Formulas I through VII, an artisan will appreciate the carbonyl covalently attached to the V group is included in the ester carboxylic acid protecting group. In some embodiments, V is an amine protecting group. Exemplary amine protecting groups include, but are not limited to, fluorenylmethyloxycarbonyl (Fmoc) or tert-butyloxycarbonyl (Boc).

In some embodiments, Q is a protecting group. In some embodiments, Q is a carboxylic acid protecting group. Exemplary carboxylic acid protecting groups include, but are not limited to, a methyl ester, a t-butyl ester, a benzyl ester, or a S-t-butyl ester. In the context of Formulas I through VII, an artisan will appreciate the carbonyl covalently attached to the Q group is included in the ester carboxylic acid protecting group. In some embodiments, Q is an amine protecting group. Exemplary amine protecting groups inc [symbol] fluorenylmethyloxycarbonyl (Fmoc) or tert-butyloxycarl

[symbol]

In some embodiments, T is C(O)OH. In some embodiments, T is H.

In some embodiments, V is C(O)OH. In some embodiments, V is H.

In some embodiments, Q is C(O)OH. In some embodiments, Q is H.

In some embodiments, T and V are each independently a peptide or an amino acid. In some embodiments, T is an amino acid or a peptide, and V is a protecting group, C(O)OH, or H. In some embodiments, V is an amino acid or a peptide, and T is a protecting group, C(O)OH, or H.

In some embodiments, T, V, and Q are each independently a peptide or an amino acid. In some embodiments, T and V are each independently a peptide or an amino acid and Q is a protecting group, C(O)OH, or H. In some embodiments, T and Q are each independently a peptide or amino acid and V is a protecting group, C(O)OH, or H. In some embodiments, V and Q are each independently a peptide or amino acid and T is a protecting group, C(O)OH, or H. In some embodiments, T is peptide or an amino acid and V and Q are each independently a protecting group, C(O)OH, or H. In some embodiments, V is peptide or an amino acids and T and Q are each independently a protecting group, C(O)OH, or H. In some embodiments, Q is peptide or an amino acid and T and V are each independently a protecting group, C(O)OH, or H.

In some embodiments when T and V are peptides, T and V may be covalently coupled as members of cyclic peptide where the cyclic peptide includes the pyrazole group, for example, as shown in Formulas I(c), II(c), III(c), IV(c), and V(c), below. As used herein the term “covalently coupled” refers to two or more groups that are linked though an intervening peptide sequence. The cyclic peptide may include, one amino acid residue, two amino acid residues, three amino acid residues, four amino acid residues, five amino acid residues, six amino acid residues, seven amino acid residues, eight amino acid residues, nine amino acid residues, ten amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues, or 20 amino acid residues.

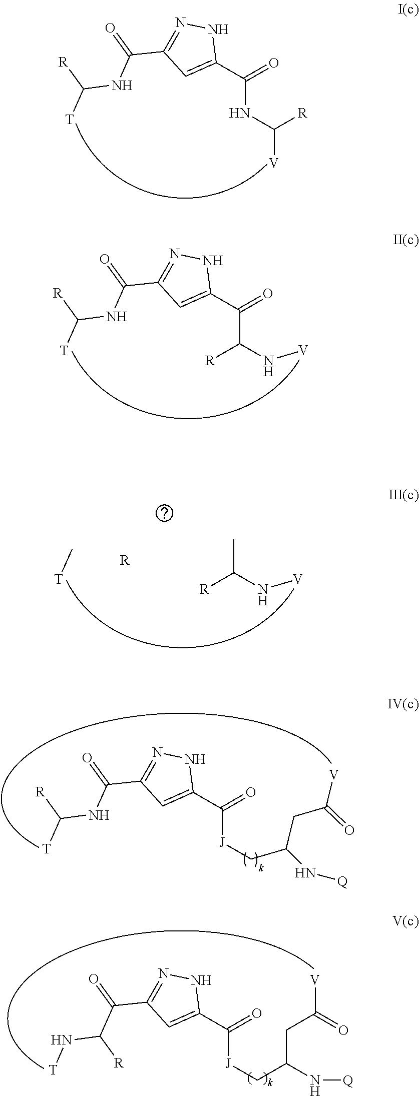

In some embodiments where T and Q are peptides, T and Q may be covalently coupled as members in a cyclic peptide that includes the pyrazole group, for example, as shown in Formulas IV(c)a and V(c)a, below. The cyclic peptide may include, one amino acid residue, two amino acid residues, three amino acid residues, four amino acid residues, five amino acid residues, six amino acid residues, seven amino acid residues, eight amino acid residues, nine amino acid residues, ten amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues, or 20 amino acid residues.

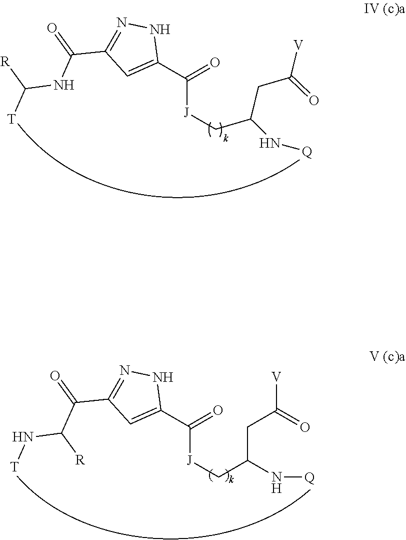

In some embodiments when V and Q are peptides, V and Q may be covalently coupled as members of cyclic peptide that does not include the pyrazole group, for example, as shown in Formula IV(c)b and V(c)b, below. The cyclic peptide may include, one amino acid residue, two amino acid residues, three amino acid residues, four amino acid residues, five amino acid residues, six amino acid residues, seven amino acid residues, eight amino acid residues, nine amino acid residues, ten amino acid residues, 11 amino a [symbol] 13 amino acid residues, 14 amino acid residues, 15 amin [symbol] 17 amino acid residues, 18 amino acid residues, 19 amino acid residues, or 20 amino acid residues.

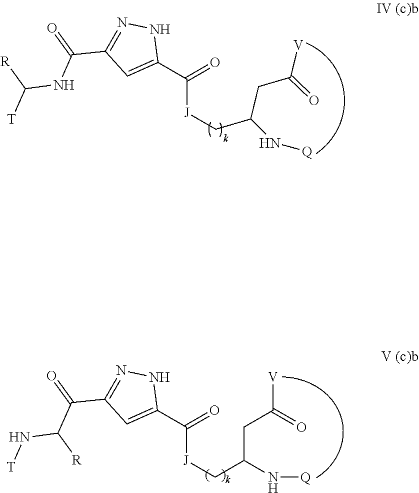

In another aspect, this disclosure describes pyrazoline-containing peptides. In some embodiments, the pyrazoline-containing peptide is of the general Formula IX or an isomer thereof. In some embodiments, the pyrazoline-containing peptide is of the general Formula X or an isomer thereof. In some embodiments, the pyrazoline-containing peptide is of the general Formula XI or an isomer thereof. In some embodiments, the pyrazoline-containing peptide is of the general Formula XII or an isomer thereof. In some embodiments, the pyrazoline-containing peptides of the general Formula XIII or an isomer thereof.

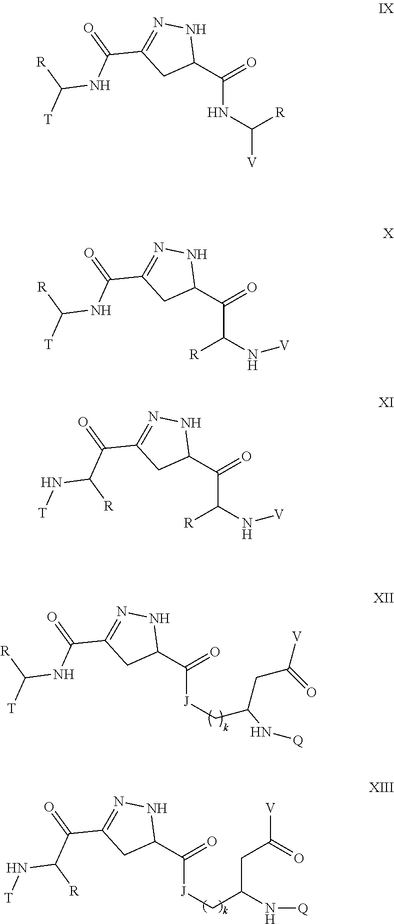

In the general Formulas IX through XIII, R, T, V, Q, and J may each be a group or structure as described above in the context of Formulas I through V and k can be as described above in the context of Formula IV and Formula V.

In some embodiments when T and V are peptides [symbol] members of a cyclic peptide where the cyclic peptide inc [symbol] as shown in Formulas, IX(c), X(c), XI(c), XII(c), and XIII(c), below. The cyclic peptide may include, one amino acid residue, two amino acid residues, three amino acid residues, four amino acid residues, five amino acid residues, six amino acid residues, seven amino acid residues, eight amino acid residues, nine amino acid residues, ten amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues, or 20 amino acid residues.

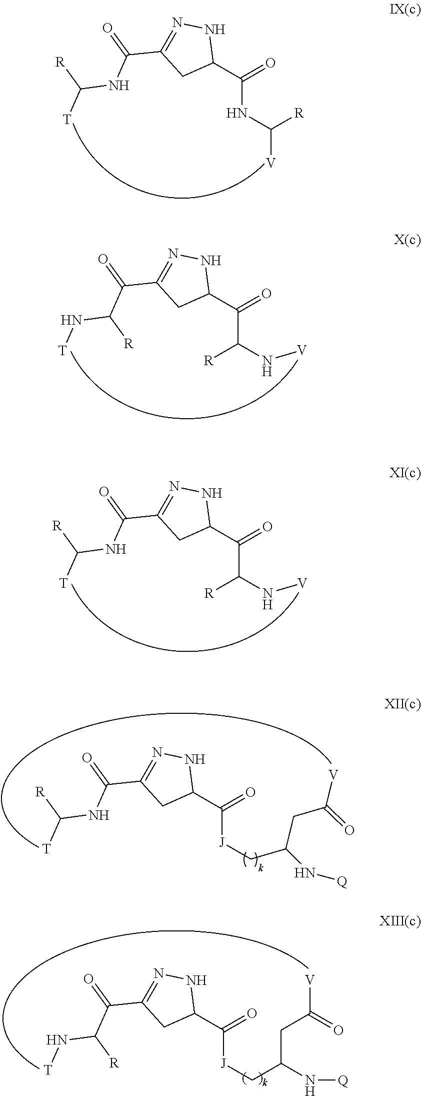

In some embodiments when T and Q are peptides, T and Q are covalently linked as members of cyclic peptide that includes the pyrazole group, for example as shown in Formulas XII(c)a and XIII(c)a, below. The cyclic peptide may include, one amino acid residue, two amino acid residues, three amino acid residues, four amino acid residues, five amino acid residues, six amino acid residues, seven amino acid residues, eight amino acid residues, nine amino acid residues, ten amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues, or 20 amino acid residues.

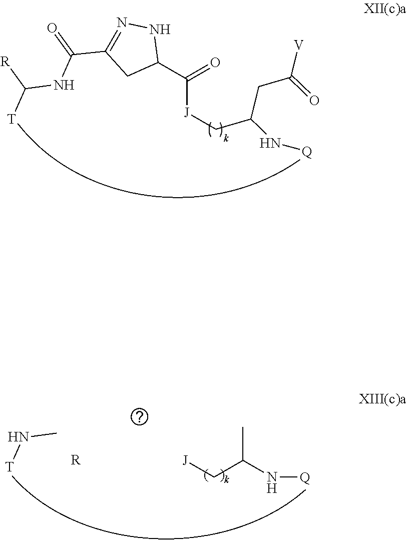

In some embodiments when V and Q are peptides, V and Q are covalently coupled as members of a cyclic peptide that does not include the pyrazole group, for example as shown in Formulas XII(c)b and XIII(c)b, below. The cyclic peptide may include, one amino acid residue, two amino acid residues, three amino acid residues, four amino acid residues, five amino acid residues, six amino acid residues, seven amino acid residues, eight amino acid residues, nine amino acid residues, ten amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues, or 20 amino acid residues.

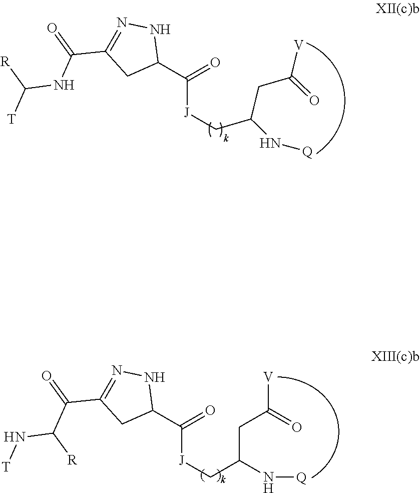

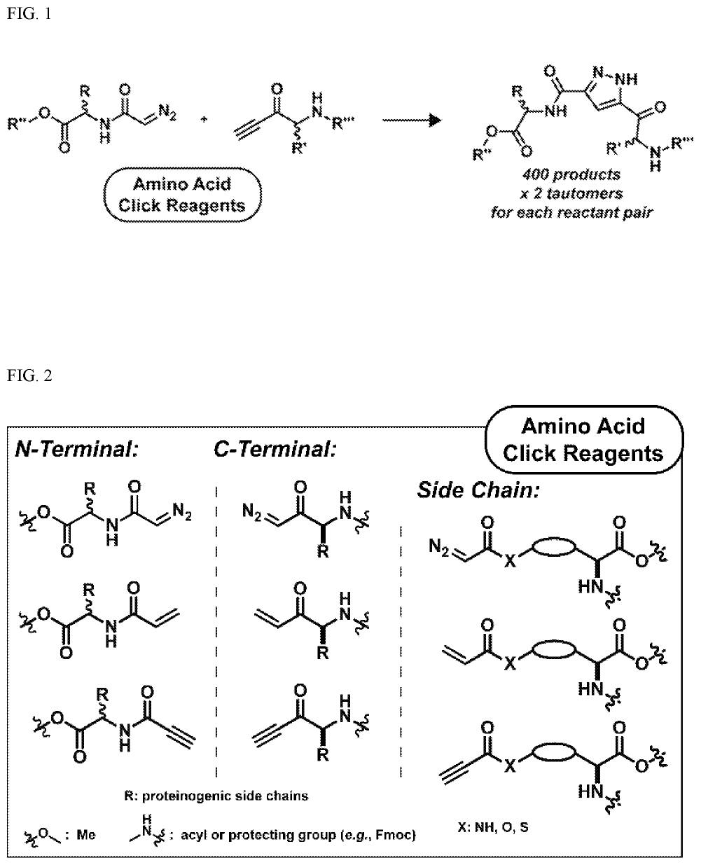

The library of pyrazole-containing peptides and a pyrazoline-containing peptides described herein exploits the diversity provided by endogenous amino acid building blocks and cycloaddition reactions. As described herein, the 1,3-dipolar “click” cycloadditions of diazo functionalized amino acids with alkene or alkyne functionalized amino acids may be used herein to generate a pyrazole-containing peptide libraries and/or a pyrazoline-containing peptide libraries. As disclosed herein, diazo functionalized amino acid can be reacted in a 1,3-dipolar cycloaddition with an alkyne functionalized amino acid to give a pyrazole-containing peptide. A representation of this reaction is shown in FIG. 1. Also, as disclosed herein, a diazo functionalized amino acid amino acid can be reacted in a 1,3-dipolar cycloaddition with alkyne functionalized amino acid to give a pyrazoline-containing peptide.

Late in the 19th century, the first 1,3-dipolar cycloaddition reaction between an electron-deficient alkene and ethyl diazoacetate was carried out. With the modern advent of “click chemistry,” cycloadditions have revolutionized the molecular sciences. Azide-alkyne cycloadditions are routine but require either metal cataly [symbol] The concept of “click chemistry” aligns with high-throug [symbol] but requisite catalysts and reaction conditions (e.g., organic solvents and heat) pose limitations in the generation of peptide libraries. Moreover, the prototypical click reaction, the copper-catalyzed azide-alkyne cycloaddition, generates a flat triazole product.

This disclosure describes catalyst-free 1,3-dipolar cycloadditions of diazo compounds and alkenes/alkynes derived from amino acids (e.g., diazo, alkyne, and/or alkene functionalized amino acids) to create structurally diverse combinatorial pyrazole-containing peptide libraries and/or pyrazoline-containing peptide libraries. Tuning of the properties of the pyrazole-containing peptide and/or the pyrazoline-containing peptides (e.g., structural, electronic, spectroscopic) can be achieved using the combinatorial approach, which also provides a means to rapidly identify and optimize promising species.

As such, in another aspect, this disclosure describes a method for synthesizing a pyrazole-containing peptide from a diazo functionalized amino acid and an alkyne functionalized amino acid. In some embodiments, the method may be used to make the pyrazole-containing peptides described herein. The method includes reacting the diazo functionalized amino acid and an alkyne functionalized amino acid in a 1,3-dipolar cycloaddition to give a pyrazole-containing peptide. In this aspect, for example, as shown in Scheme 1:

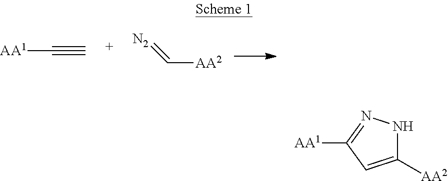

the alkyne functionalized amino acid is reacted with the diazo functional amino acid to produce a pyrazole-containing peptide, where AA¹ and AA² are each independently an amino acid of the Formulas A, B, or C.

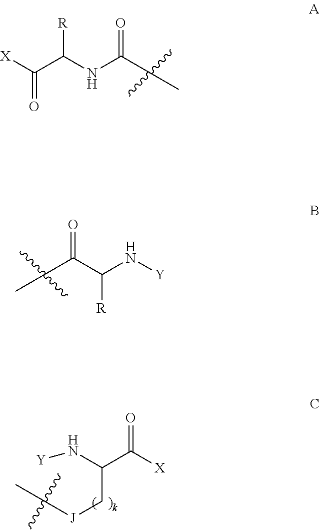

Formulas A, B, and C are configurations of amine [symbol] and/or alkene functional group (e.g., diazo functionalized [symbol] amino acid). The alkene functional group may be used to make a pyrazoline-containing peptide as discussed elsewhere herein. For example, Formula A is an N-terminal functionalized amino acid. Formula B is a C-terminal functionalized amino acid. Formula C is a side chain functional amino acid.

Compounds of Formulas A, B, and C may be synthesized using methods known in the art. For example, diazo, alkyne, and alkene functionalized amino acids of Formulas A and C may be synthesized by reacting the N-terminus or a nucleophilic side chain (Cys, Lys, Ser, Thr, Hyp, etc.) of an amino acid with the corresponding N-hydroxysuccinimidyl (NHS) compound functionalized with a diazo group, alkene group, or alkyne group. C-terminal functionalized amino acids may be synthesized via well-established synthetic methods, e.g., via the AlCl₃-promoted addition of vinyl and alkynyl silanes to the acyl chloride generated from each amino acid.

In the Formulas A and B, R is the side chain or protected side chain of an amino acid. Exemplary reactive groups found on amino acid side chains and suitable amino acid side chain protecting groups are described elsewhere herein.

In some embodiments, the amino acid side chain is the side chain of any proteogenic amino acid. In some embodiments, the amino acid side chain is the side chain of any unnatural amino acid. In some embodiments, the amino acid side chain may be the side chain or protected side chain of Gly, Ala, Val, Ile, Leu, Met, Phe, Tyr, Trp, Pro, Cys, Lys, Ser, Thr, Asn, Gln, Arg, His, Asp, Glu, Hyp (hydroxyproline), chloroproline, aminoproline, phosphoserine, phosphotyrosine, fluorotyrosine, fluorotryptophan, bromotryptophan, citrulline, norvaline, norleucine, N-methyl lysine (mono, di, or tri), N-methyl arginine (mono, di, or tri), 2,4-diaminobutyric acid, 2,3-diaminobutyric acid, penicillamine, fluoroproline (Flp), selenocysteine (Sec), ornithine (Orn), or a derivative of any of the foregoing. Any enantiomer or diastereomer of the amino acid side chain is contemplated.

In some embodiments, the amino acid side chain is the side chain of Gly. In some embodiments, the amino acid side chain is the side chain of Ala. In some embodiments, the amino acid side chain is the side chain of Ile. In some embodiments, the amino acid side chain is the side chain of Val. In some embodiments, the amino acid side chain is the side chain of Leu. In some embodiments, the amino acid side chain is the side chain or protected side chain of Met. In some embodiments, the amino acid side chain is the side chain of Phe. In some embodiments, the amino acid side chain is the side or pro [symbol] embodiments, the amino acid side chain is the side chain [symbol] embodiments, the amino acid side chain is the side chain or protected side chain of Pro. In some embodiments, the amino acid side chain is the side chain or protected side chain of Cys. In some embodiments, the amino acid side chain is the side chain or protected side chain of Lys. In some embodiments, the amino acid side chain is the side chain or protected side chain of Ser. In some embodiments, the amino acid side chain is the side chain or protected side chain of Thr. In some embodiments, the amino acid side chain is the side chain or protected side chain of Asn. In some embodiments, the amino acid side chain is the side chain or protected side chain of Gln. In some embodiments, the amino acid side chain is the side chain or protected side chain of Arg. In some embodiments, the amino acid side chain is the side chain or protected side chain of His. In some embodiments, the amino acid side chain is the side chain or protected side chain of Asp. In some embodiments, the amino acid side chain is the side chain or protected side chain of Glu. In some embodiments, the amino acid side chain is the side chain or protected side chain of Hyp. In some embodiments, the amino acid side chain is the side chain or protected side chain of Flp. In some embodiments, the amino acid side chain is the side chain or protected side chain of Sec. In some embodiments, the amino acid side chain is the side chain or protected side chain of Orn. In some embodiments, the amino acid side chain is the side chain or protected side chain of chloroproline. In some embodiments, the amino acid side chain is the side chain or protected side chain of aminoproline. In some embodiments, the amino acid side chain is the side chain or protected side chain of phosphoserine. In some embodiments, the amino acid side chain is the side chain or protected side chain of phosphotyrosine. In some embodiments, the amino acid side chain is the side chain or protected side chain of fluorotyrosine. In some embodiments, the amino acid side chain is the side chain or protected side chain of fluorotryptophan. In some embodiments, the amino acid side chain is the side chain or protected side chain of bromotryptophan. In some embodiments, the amino acid side chain is the side chain or protected side chain of citrulline. In some embodiments, the amino acid side chain is the side chain or protected side chain of norvaline. In some embodiments, the amino acid side chain is the side chain or protected side chain of norleucine. In some embodiments, the amino acid side chain is the side chain or protected side chain of N-methyl lysine (mono, di, or tri). In some embodiments, the amino acid side chain is the side chain or protected side chain of N-methyl arginine (mono, di, or tri). In some embodiments, the amino acid side chain is the side chain or protected side chain of, 2,4-diaminobutyric acid. In some embodiments, the amino acid side chain is the side chain or protected side chain of 2,3 [symbol] embodiments, the amino acid side chain is the side chain

[symbol]

In Formulas A, B, and C, X may be an amino acid, a peptide, a protecting group, H, or C(O)OH.

In some embodiments, X is an amino acid or a peptide. In embodiments when X is an amino acid, the amino acid is the C-terminal amino acid. In embodiments where X is a peptide, the peptide extends in the C-terminal direction such that the last amino acid in the peptide is the C-terminal amino acid. In some embodiments when X is an amino acid, the C-terminus may be protected via a protecting group. In some embodiments when X is an amino acid, the side chain of X may be a protected via a protecting group (e.g., a protected side chain). In some embodiments when X is an amino acid, the C-terminus may be protected via a protecting group, and the side chain of X may be a protected via a protecting group. In some embodiments when X is a peptide, the C-terminus of the peptide may be protected via a protecting group. In some embodiments when X is a peptide, the side chain of one or more of the amino acids in the peptide may be protected via a protecting group. In some embodiments when X is a peptide, the C-terminus of the peptide may be protected via protecting group and the side chain of one or more of the amino acids in the peptide may be protected via a protecting group. Suitable side chain and C-terminus protecting groups are known in the field and discussed elsewhere herein.

In some embodiments, X is a protecting group, particularly a carboxylic acid protecting group. Exemplary carboxylic acid protecting groups include, but are not limited to, a methyl ester, a t-butyl ester, a benzyl ester, or a S-t-butyl ester. In the context of Formulas A, B, and C, an artisan will appreciate the carbonyl covalently attached to the X group is included in the ester carboxylic acid protecting group. In some embodiments, following the 1,3-dipolar cycloaddition reaction, the protecting group may be removed to expose a carboxylic acid using methods known in the art.

In some embodiments, X is C(O)OH.

In Formulas A, B, and C, Y may be an amino acid, a peptide, a protecting group, or H.

In some embodiments, Y is an amino acid or a peptide. In embodiments when Y is an amino acid, the amino acid is the N-terminal amino acid. In embodiments where Y is peptide, the peptide extends in the N-terminal direction such that the last amino acid in the peptide is the N-terminal amino acid. In some embodiments when Y is an amino acid, the N-terminus may be protected via a protecting group. In some embodiments when Y is an amino acid, the side chain of Y may be a protected via a protecting group (e.g., a protected side chain). In some embodiments when Y is an amino acid, the N-terminus n [symbol] and the side chain of Y may be a protected via a protectir [symbol] Y is a peptide, the N-terminus of the peptide may be protected via a protecting group. In some embodiments when Y is a peptide, the side chain of one or more of the amino acids in the peptide may be protected via a protecting group. In some embodiments when Y is a peptide, the N-terminus of the peptide may be protected via protecting group and the side chain of one or more of the amino acids in the peptide may be protected via a protecting group. Suitable side chain and N-terminus protecting groups are known in the field and discussed elsewhere herein.

In some embodiments, Y is a protecting group, particularly an amine protecting group. Exemplary amine protecting groups include, but are not limited to, fluorenylmethyloxycarbonyl (Fmoc) or tert-butyloxycarbonyl (Boc). In some embodiments, following the 1,3-dipolar cycloaddition reaction, the protecting group may be removed to an amine using methods known in the art.

In some embodiments, Y is H.

In Formula C, J is a functionalized amino acid side chain. The amino acid side chain is functionalized such that it is covalently bonded to a terminal alkyne moiety or a terminal diazo moiety. In some embodiments J is of Formula J(I), (JII), J(III), or J(IV):

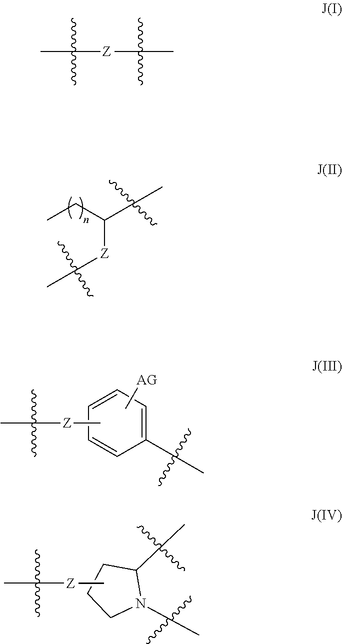

In Formulas J(I), J(II), J(III), and J(IV), Z is covalently bonded to a terminal alkyne moiety or a terminal diazo moiety. In Formulas of J(I), J(II), J(III), and J(IV), Z is nitrogen (N), oxygen (O), sulfur (S), selenium (Se) or the protonated or alkylated equivalent. In some embodiments, Z is N. In some embodiments, Z is NH. In some embodiments, Z is NH₂. In some embodiments, Z is O. In some embodiments, Z is S. In some embodiments, Z is SH. In some embodiments, Z is Se.

In the Formula J(III), AG is a functional group su [symbol] alkyl. In some embodiments AG is a halogen. In some er [symbol] embodiments, AG is an alkyl.

In Formula C, k is 0, 1, 2, 3, 4, 5, 6, 7, or 8. In some embodiments, k is 0. In some embodiments, k is 1. In some embodiments, k is 2. In some embodiments, k is 3. In some embodiments, k is 4. In some embodiments, k is 5. In some embodiments, k is 6. In some embodiments, k is 7. In some embodiments, k is 8.

In the Formula J(II), n is 0, 1, 2, 3, 4, 5, 6, 7, or 8. In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8.

In some embodiments when J is J(II), Z is O, n is 1, and k is 1. In some embodiments when J is J(I), k is 1 and Z is O. In some embodiments when J is J(I), k is 1 and Z is S.

In some embodiments when J is J(II), k is 1, n is 1 and Z is S. In some embodiments when J is J(II), k is 2, n is 1 and Z is S. In some embodiments when J is J(II), k is 1, n is 1 and Z is Se. In some embodiments when J is J(II), k is 2, n is 1 and Z is Se. In some embodiments when J is J(II), k is 1, n is 1 and Z is O. In some embodiments when J is J(II), k is 2, n is 1 and Z is O.

In some embodiments when J is J(III), k is 1 and Z is O. In some embodiments when J is J(III), k is 1 and Z is NH. In some embodiments when J is J(III), k is 1 and Z is NH₂. In some embodiments when J is J(III), k is 1 and Z is S. In some embodiments when J is J(III), k is 1 and Z is SH.

An artesian will appreciate that in Formula J(IV), k is 0 and the nitrogen in the five membered ring is the nitrogen that is covalently attached to Y in Formula C. In some embodiments when J is J(IV), Z is O. In some embodiments when J is J(IV), Z is SH. In some embodiments when J is J(IV), Z is NH₂. In some embodiments when J is J(IV), Z is NH.

In some embodiments, the reaction conditions are mild. In some embodiments, the reaction conditions include an aqueous environment. In some embodiments, the reaction conditions include ambient temperature. In some embodiments, the reaction conditions include ambient pressure. In some embodiments, the reaction conditions include less than a catalytic amount of a catalyst. In some embodiments, the reaction conditions include less than a catalytic amount of a metal-based catalyst. In some embodiments, the reaction conditions may include any combination of the above-mentioned conditions.

In some embodiments, the pyrazole-containing pe [symbol] acyclic and/or cyclic peptides. This can be done by carry [symbol] on a resin, using common structural motifs (e.g., β-turn, α-helix, β-sheet, PPII helix, etc.), peptide stapling (sidechain-to-sidechain, sidechain-to-tail, head-to-tail, etc.), and/or libraries of di- and/or tri-amino acid building blocks.

In some embodiments, trimethylsilyldiazomethane (TMS diazomethane), which is typically used as a methylating agent and as a source of CH₂ groups, may be reacted with an alkyne functionalized amino acids, thereby allowing functionalization of TMS-substituted pyrazoles containing peptides.

In some embodiments, the method may include removing one or more protecting groups after the completing the 1,3-cycloaddition reaction. For example, in some embodiments, the method may include removing the protecting group on one or more protected amino acid side chains. In some embodiments, the method may include removing the protecting group on one or more C-terminal amino acids or on one or more N-terminal amino acids. In some embodiments, the method may include removing the protecting group on one or more N-terminal amino acids. In some embodiments, the method may include removing any of the above-mentioned protecting groups.

In some embodiments, the pyrazole-containing peptides can be generated to determine optimal conditions for combinatorial syntheses. This can be accomplished using the absorbance and emission of each synthesized building block, and monitoring reactivity via absorbance and/or fluorescence utilizing a plate reader. Product formation can be confirmed via HPLC/MS and, in a number of cases, fully characterize products via ¹H and ¹³C NMR, and HRMS. While spectral overlap may pose challenges for certain reactant pairs, the ability to screen all amino acid combinations can provide insights into general reaction kinetics, allowing for the determination of typical reaction times. Reactions can be monitored via infrared spectroscopy, if necessary.

In another aspect, this disclosure describes a method for synthesizing pyrazoline-containing peptides from a diazo functionalized amino acid and an alkene functionalized amino acid. In some embodiments, the method may be used to make the pyrazoline-containing peptides described elsewhere herein. The method includes a 1,3-dipolar cycloaddition of the diazo functionalized amino acid and the alkene functionalized amino acid to give a pyrazoline-containing peptide. In this aspect, for example, as shown in Scheme 2:

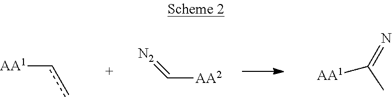

the alkene functional amino acid is reacted with the diazo functional amino acid to produce a pyrazole-containing peptide, where AA¹ and AA² are each independently an amino acid of Formula A, B, or C.

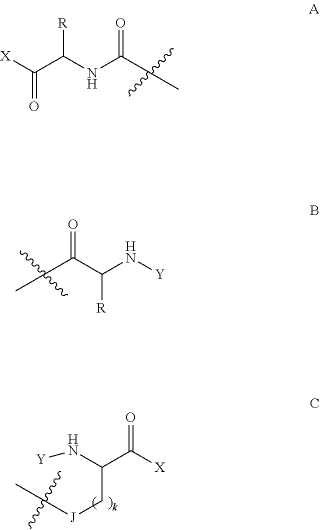

Formulas A, B, and C may be any structure, formula, or configuration as described relative to Scheme 1.

In some embodiments, the reaction conditions are mild. In some embodiments, the reaction conditions include an aqueous environment. In some embodiments, the reaction conditions include ambient temperature. In some embodiments, the reaction conditions include ambient pressure. In some embodiments, the reaction conditions include less than a catalytic amount of a catalyst. In some embodiments, the reaction conditions include less than a catalytic amount of a metal-based catalyst. In some embodiments, the reaction conditions may include any combination of the above-mentioned conditions.

In some embodiments, the pyrazoline-containing peptides may be incorporated into larger acyclic and/or cyclic peptides. This can be done by carrying out the 1,3-cycloaddition reaction on a resin, using common structural motifs (e.g., β-turn, α-helix, β-sheet, PPII helix, etc.), peptide stapling (sidechain-to-sidechain, sidechain-to-tail, head-to-tail, etc.), and/or libraries of di- and/or tri-amino acid building blocks.

In some embodiments, trimethylsilyldiazomethane (TMS diazomethane), which is typically used as a methylating agent and as a source of CH₂ groups, may be reacted with an alkene functionalized amino acids, thereby allowing functionalization of TMS-substituted pyrazolines containing peptides.

In some embodiments, the method may include re [symbol] after the completing the 1,3-cycloaddition reaction. For e [symbol] method may include removing the protecting group on one or more protected amino acid side chains. In some embodiments, the method may include removing the protecting group on one or more C-terminal amino acids or on one or more N-terminal amino acids. In some embodiments, the method may include removing the protecting group on one or more N-terminal amino acids. In some embodiments, the method may include removing any of the above-mentioned protecting groups.

In some embodiments, the pyrazoline-containing peptides can be generated to determine optimal conditions for combinatorial syntheses. This can be accomplished using the absorbance and emission of each synthesized building block, and monitoring reactivity via absorbance and/or fluorescence utilizing a plate reader. Product formation can be confirmed via HPLC/MS and, in a number of cases, fully characterize products via ¹H and ¹³C NMR, and high resolution mass spectrometry. While spectral overlap may pose challenges for certain reactant pairs, the ability to screen all amino acid combinations can provide insights into general reaction kinetics, allowing for the determination of typical reaction times. Reactions can be monitored via infrared spectroscopy, if necessary.

In another aspect, this disclosure describes a library of pyrazole-containing peptides and/or a library of pyrazoline-containing peptides. As used here “library” refers to a plurality of compounds in which at least two of the compounds in the plurality of compounds are different.

In some embodiments a library of pyrazole-containing peptides may be produced using the methods disclosed herein. In some embodiments the libraries of pyrazoline-containing peptides may be produced using the methods disclosed herein.

This disclosure exploits the properties of heterocycles produced from the 1,3-dipolar cycloaddition of amino acid-derived reagents (e.g., diazo, alkyne, and/or alkene functionalized amino acids) for use in forming drug compounds. Specifically, this disclosure provides methods for synthesizing synthetic pyrazole-containing peptides and pyrazoline-containing peptides, which can be used in high-throughput screening of combinatorial “click” libraries for drug development. Significantly, this provides new chemical strategies to generate libraries of synthetic peptides that can harness unique chemical properties for biomedicine and the overall improvement of human health.

In some embodiments, the library of pyrazole-cor [symbol] pyrazoline-containing peptides may be screened against 1 [symbol] activity.

The ability of diazo functionalized amino acids to undergo 1,3-dipolar cycloadditions with alkyne and/or alkyne functionalized amino acids in aqueous conditions allows one to generate diverse libraries of pyrazole-containing peptides and pyrazoline-containing peptides. For example, with various amino acid-derived library components, such as N-terminal diazo functionalized amino acids and N-terminal alkyne functionalized amino acids derived from Gly, Val, or Pro, quantitative conversion to the pyrazole-containing peptide can be accomplished. This can be accomplished using both N-terminal and C-terminal diazo, alkyne, and/or alkene functionalized amino acids, as well as side chain functionalized amino acids.

To systematically develop pyrazole-containing peptide libraries and/or pyrazoline-containing peptide libraries, sets of 1,3-dipolar cycloaddition reactants derived from amino acids (e.g., diazo, alkyne, and alkene functionalized amino acids) can be synthesized as discussed elsewhere herein. Rapid generation of vastly diverse pyrazole-containing peptide libraries and/or pyrazoline-containing peptide libraries can be afforded by various 3-dipolar cycloaddition reactions using a pair of a diazo functionalized amino acid and an alkyne functionalized amino acid or an alkene functionalized amino acid. Each reactant pair generates 400 pyrazole-containing peptides or pyrazoline-containing peptide, which can be further functionalized and/or screened for biological activity.

In some embodiments, for each amino acid, a set of N-terminal, C-terminal, and side-chain functionalized diazo compounds, alkenes, and alkynes can be generated and 1,3-dipolar cycloadditions reaction carried out to generate pyrazole-containing peptides and/or pyrazoline-containing peptides. In certain embodiments, with approximately 200 diazo, alkyne or alkene functionalized amino acids (approximately 60 N-terminal, approximately 120 C-terminal, and approximately 20 side chain functionalized amino acids), over 7,000 cycloadducts can be formed.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endr [symbol] within that range (e.g., 1 to 5 includes 1, 1.5, 2, 2.75, 3, 3

[symbol]

In the preceding description, particular embodiments may be described in isolation tor clarity. Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be conducted in any feasible order. And, as appropriate, any combination of two or more steps may be conducted simultaneously.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The invention is defined in the claims. However, below there is provided a non-exhaustive listing of non-limiting exemplary embodiments. Any one or more of the features of these embodiments may be combined with any one or more features of another example, embodiment described herein.

### EXEMPLARY EMBODIMENTS

Embodiment 1 is a pyrazole-containing peptide of one of formulas,

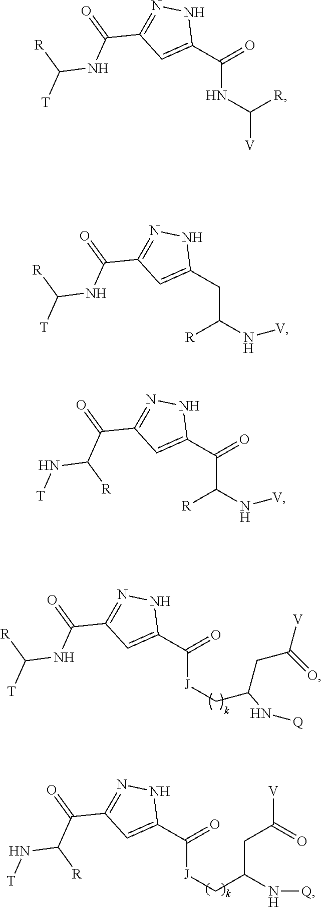

or an isomer thereof, wherein: each R is independently an amino acid side chain; T, V, and Q are each independently an amino acid, a peptide, an amine, an OH, or a protecting group; k is 0, 1, 2, 3, 4, 5, 6, 7, or 8; and J is one of the formulas

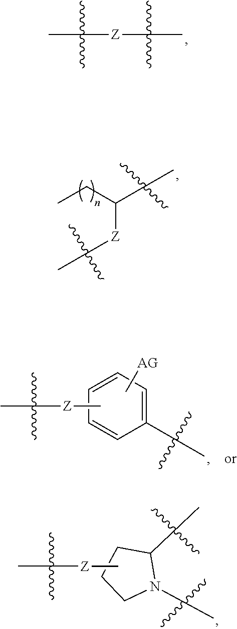

wherein each Z is independently O, N, NH, S, SH, or Se; n is 0, 1, 2, or 3; and AG is a halogen or alkyl.

Embodiment 2 is a pyrazoline-containing peptide of one of formulas,

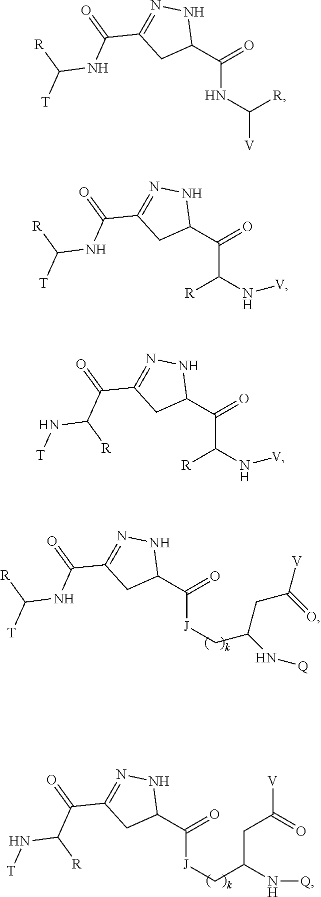

or an isomer thereof wherein: each R is independently an amino acid side chain; T, V, and Q are each independently an amino acid, a peptide, an amine, an OH, or a protecting group; k is 0, 1, 2, 3, 4, 5, 6, 7, or 8; and J is one of the formulas

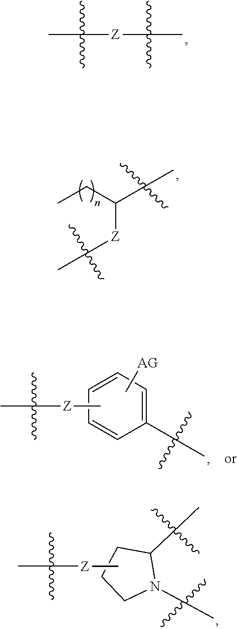

wherein each Z is independently O, N, NH, S, SH, or Se; n is 0, 1, 2, or 3; and AG is a halogen or alkyl.

Embodiment 3 is any one of Embodiments 1-2, wherein T and V are peptides.

Embodiment 4 is any one of Embodiments 1-3, wherein T, V, and Q are peptides.

Embodiment 5 is any one of Embodiments 1-4, wherein T and Q are peptides.

Embodiment 6 is any one of Embodiments 1-5, wherein V and Q are peptides.

Embodiment 7 is any one of Embodiments 1-6, wherein T and V are covalently coupled through a cyclic peptide.

Embodiment 8 is any one of Embodiments 1-7, wherein T and Q are covalently coupled through a cyclic peptide.

Embodiment 9 is any one of Embodiments 1-8, wherein V and Q are covalently coupled through a cyclic peptide.

Embodiment 10 is any one of Embodiments 1-9, wherein T is an amino acid and Vis an amino acid.

Embodiment 11 is any one of Embodiments 1-10, wherein Tis an amino acid and Vis H or C(O)OH.

Embodiment 12 is any one of Embodiments 1-11, wherein V is an amino acid and Tis H or C(O)OH.

Embodiment 13 is any one of Embodiments 1-12, wherein Tis an amino acid, Vis an amino acid and Q is H or C(O)OH.

Embodiment 14 is any one of Embodiments 1-13, wherein Tis an amino acid, Q is an amino acid and V is H or C(O)OH.

Embodiment 15 is any one of Embodiments 1-14, wherein V is an amino acid, Q is an amino acid and T is H or C(O)OH.

Embodiment 16 is any one of Embodiments 1-15 [symbol] C(O)OH, and Q is H or C(O)OH.

Embodiment 17 is any one of Embodiments 1-16, wherein V is an amino acid, T is H or C(O)OH, and Q is H or C(O)OH.

Embodiment 18 is any one of Embodiments 1-17, wherein Q is an amino acid, T is H or C(O)OH, and V is H or C(O)OH.

Embodiment 19 is any one of Embodiments 1-18, wherein each R is independently the side chain of a proteogenic amino acid.

Embodiment 20 is any one of Embodiments 1-19 further including a method for synthesizing a pyrazole-containing peptide or a pyrazoline-containing peptide comprising: reacting an alkyne functionalized amino acid of formula

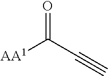

or a alkene functionalized amino acid of formula

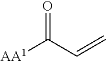

with a diazo functionalized amino acid of formula

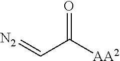

under a set of reaction conditions,
wherein AA¹ and AA² are each independently one of formulas:

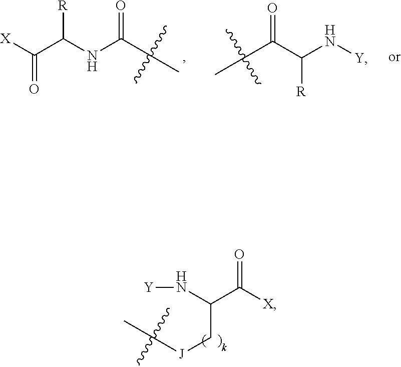

wherein: each R is independently an amino acid side chain; X and Y are each independently C(O)OH, H, an amine protecting group or a carboxylic acid protecting group; k is 0, 1, 2, 3, or 4; and J is of the of the general formula:

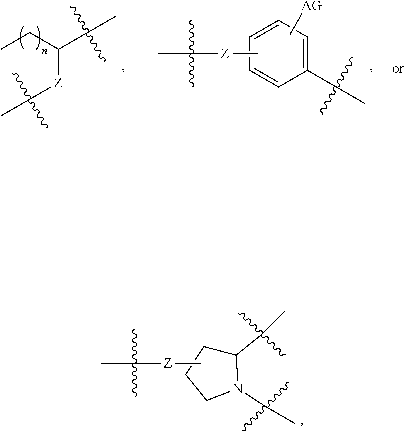

wherein: each Z is independently O, N, NH, S, SH, or Se; n is 0, 1, 2, 3, or 4; and AG is a halogen or alkyl.

Embodiment 21 is any one of Embodiments 1-20, wherein the the pyrazole-containing peptide is of formula

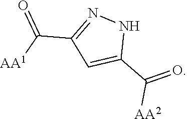

Embodiment 22 is any one of Embodiments 1-21 [symbol] peptide of formula

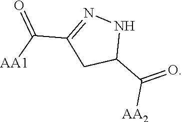

Embodiment 23 is any one of Embodiments 1-21, wherein AA¹ and AA² are

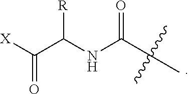

Embodiment 24 is any one of Embodiments 1-23, wherein AA¹ is

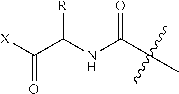

### and AA² is

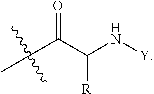

Embodiment 25 is any one of Embodiments 1-24, wherein AA¹ and AA² are

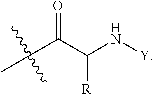

Embodiment 26 is any one of Embodiments 1-25, wherein AA¹ is

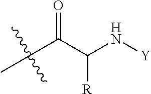

### and AA² is

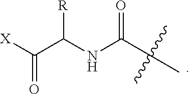

Embodiment 27 is any one of Embodiments 1-26, wherein AA¹ is

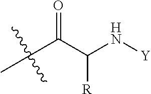

### and AA² is

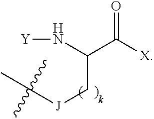

Embodiment 28 is any one of Embodiments 1-27, wherein AA is [symbol] AA² is

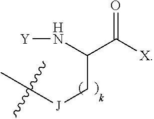

Embodiment 29 is any one of Embodiments 1-28, wherein AA¹ is

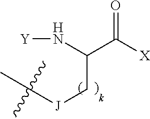

### and AA² is.

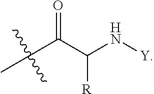

Embodiment 30 is any one of Embodiments 1-29, wherein AA¹ is

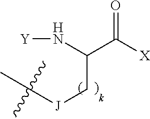

### and AA² is

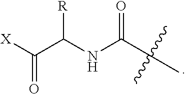

Embodiment 31 is any one of Embodiments 1-30, wherein AA¹ and AA² are

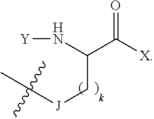

Embodiment 32 is any one of Embodiments 1-31, wherein each R is the side chain of a proteogenic amino acid.

Embodiment 33 is any one of Embodiments 1-32, wherein each R is the side chain of an unnatural amino acid.

Embodiment 34 is any one of Embodiments 1-33, wherein the reacting conditions comprise one or more of an aqueous environment, catalyst-free environment, ambient temperature, and ambient pressure.

Embodiment 35 is any one of Embodiments 1-34, further comprising removing one or more protecting groups.

Embodiment 36 is any one of Embodiments 1-35 [symbol] a library of pyrazole-containing peptides comprising repe [symbol] Embodiments 1-35 to produce a plurality of pyrazole-containing peptides.

Embodiments 37 is any one of Embodiments 1-36, further including a method of making a library of pyrazoline-containing peptides comprising repeating the method of any one of Embodiments 1-35 to produce a plurality of pyrazoline-containing peptides.

Embodiment 38 is any one of Embodiments 1-37, further including a method of making a library of synthetic amino acid-derived compounds, the method comprising providing building blocks comprising a set of diazo-functional amino acids and a set of alkene- or alkyne-functional amino acids; and reacting one of the set of diazo-functional amino acids with one of the set of alkene- or alkyne-functional amino acids under conditions effective to form a pyrazole- or pyrazoline-containing peptide cycloadduct of the library; and repeating the reacting step with a different diazo-functional amino acid and a different alkene- or alkyne-functional amino acid under conditions effective to form a different pyrazole- or pyrazoline-containing peptide cycloadduct of the library.

Embodiment 39 is any one of Embodiments 1-38, wherein the diazo-functional amino acid is an N-terminal diazo-functional amino acid (i.e., an N-terminal amino acid-based diazo compound or a diazoacetamide), a C-terminal diazo-functional amino acid (C-terminal amino acid-based diazo compound), or a side chain diazo-functional amino acid.

Embodiment 40 is any one of Embodiments 1-39, wherein the alkene- or alkyne-functional amino acid is an N-terminal alkene- or alkyne-functional amino acid (i.e., an N-terminal amino acid-based alkene or alkyne compound), a C-terminal alkene- or alkyne-functional amino acid (C-terminal amino acid-based alkene or alkyne compound), or a side chain alkene or alkyne-functional amino acid.

Embodiment 41 is any one of Embodiments 1-40, wherein the building blocks comprise 60 N-terminal, 120 C-terminal, and 20 side-chain functionalized building blocks.

Embodiment 42 is any one of Embodiments 1-41, wherein the library comprises over 7,000 cycloadducts.

Embodiment 43 is any one of Embodiments 1-42, wherein the conditions comprise aqueous, catalyst-free 1,3-dipolar cycloaddition under ambient conditions (e.g., room temperature).

Embodiment 44 is any one of Embodiments 1-43, wherein the pyrazole- or pyrazoline-containing cycloadducts form 3-dimensional scaffolds for drug discovery.

Embodiment 45 is any one of Embodiments 1-44 [symbol] Val, Ile, Leu, Met, Phe, Tyr, Trp, Pro, Cys, Lys, Ser, Thr [symbol] Flp, Sec, Orn, or other common derivatives, and their enantiomers/diastereomers.

Embodiment 46 is any one of Embodiments 1-45, wherein the alkene- or alkyne-functional amino acids are reacted with trimethylsilyldiazomethane (TMS diazomethane).

Embodiment 47 is any one of Embodiments 1-46, wherein the pyrazole- or pyrazoline-containing cycloadduct is incorporated into larger cyclic or acyclic peptides.

The present invention is illustrated by the following examples. It is to be understood that the particular examples, materials, amounts, and procedures are to be interpreted broadly in accordance with the scope and spirit of the invention as set forth herein.

### EXAMPLES

These Examples are merely for illustrative purposes and are not meant to be overly limiting on the scope of the appended claims. Notwithstanding that the numerical ranges and parameters setting forth the broad scope of this disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

### Combinatorial Synthesis of Pyrazoles:

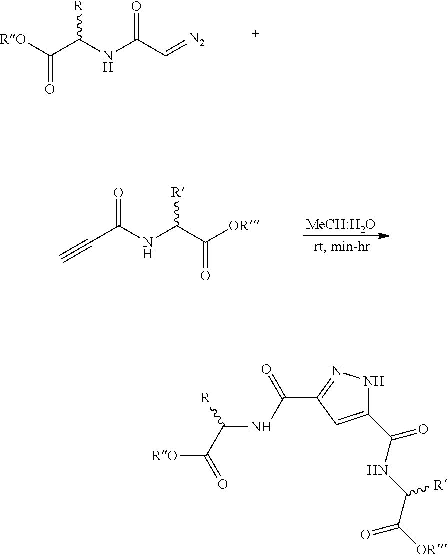

N-terminal Diazo+N-terminal alkyne: A solution of N-terminal Diazo (1 equiv.) and N-terminal alkyne (1 equiv.) in 1:1 MeCN:H₂O (0.44 M) (although DMSO could be used) was stirred at room temperature. The reaction was monitored by TLC. Upon completion, the reaction mixture was dried under high vacuum to give the pyrazole cycloadduct.

Methyl (5-(((S)-1-methoxy-1-oxo-3-phenylpropan-2-yl)carbamoyl)-1H-pyrazole-3-carbonyl)-L-valinate: Methyl (5-(((S)-1-methoxy-1-oxo-3-phenylpropan-2-yl)carbamoyl)-1H-pyrazole-3-carbonyl)-L-valinate was obtained in 73% yield. ¹H NMR (500 MHz, CDCl₃) δ 7.63 (br.s, 1H), 7.29-7.18 (m, 3H), 7.16-7.10 (m, 3H), 6.94 [symbol] (dd, J=9.0, 5.4 Hz, 1H), 3.76 (s, 3H), 3.75 (br.s, 3H), 3. [symbol] 1.00 (d, J=6.8 Hz, 3H), 0.97 (d, J=6.9 Hz, 3H). HRMS (ESI) calcd. tor C₂₁H₂₇N₄O₆ [M+H]⁺ 431.1931, found 431.1925.

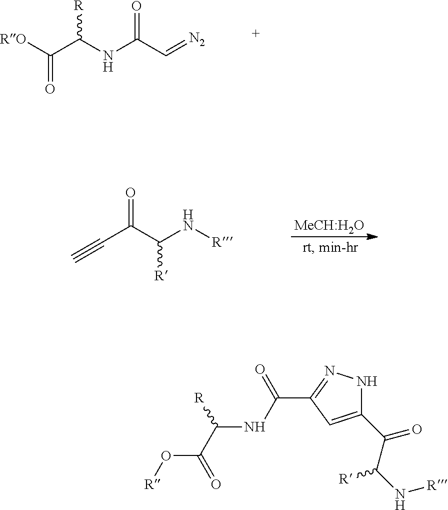

N-terminal Diazo+C-terminal alkyne: A solution of N-terminal Diazo (1 equiv.) and C-terminal alkyne (1 equiv.) in 1:1 MeCN:H₂O (0.44 M) will be stirred at room temperature. The reaction will be monitored by TLC. Upon completion, the reaction mixture will be dried under high vacuum to give the pyrazole cycloadduct.

### Synthesis of Pyrazolines:

N-terminal Diazo+N-terminal alkene: A solution of N-terminal Diazo (1 equiv.) and N-terminal alkene (1 equiv.) in 1:1 MeCN:H₂O (0.44 M) will be stirred at room temperature. The reaction will be monitored by TLC. Upon completion, the reaction mixture will be dried under high vacuum to give the pyrazoline cycloadduct(s).

Dimethyl 2,2′-((4,5-dihydro-1H-pyrazole-3,5-dicarbonyl)bis(azanediyl))(2S,2′S)-bis(3-phenylpropanoate): Dimethyl 2,2′-((4,5-dihydro-1H-pyrazole-3,5-dicarbonyl)bis(azanediyl))(2S,2'S)-bis(3-phenylpropanoate) was obtained in 71% yield. 1H NMR (500 MHz, DMSO) δ 8.3 (s, 1H), 8.1 (s, 1H), 7.9 (s, 1H), 7.3-7.2 (m, 10H), 4.6-4.5 (m, 1H), 4.5-4.5 (m, 1H), 4.3-4.2 (m, 1H), 3.6 (s, 3H), 3.6 (s, 3H), 3.1-3.0 (m, 4H), 3.0-2.9 (m, 1H), 2.7-2.5 (m, 1H). ¹³C NMR (126 MHz, DMSO) δ 171.9, 171.7, 171.4, 161.5, 143.5, 137.5, 137.0, 129.1, 129.0, 128.3, 128.2, 126.6, 126.5, 61.4, 53.5, 53.4, 52.0, 52.0, 36.5, 36.1, 35.6(−).

N-terminal Diazo+C-terminal alkene: A solution of N-terminal Diazo (1 equiv.) and C-terminal alkene (1 equiv.) in 1:1 MeCN:H₂O (0.44 M) will be stirred at room temperature. The reaction will be monitored by TLC. Upon completion, the reaction mixture will be dried under high vacuum to give the pyrazoline cycloadduct(s).

### Synthesis of Building Blocks:

### Synthesis of N-Terminal Diazo-Building Blocks; Method 1:

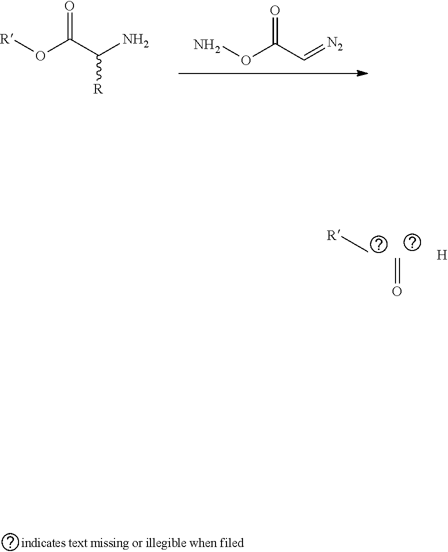

Preparation of N-Diazo-XX-OMe. N-Diazo-XX-OMe was prepared following a modified procedure of Chou and Raines (J. Am. Chem. Soc. 2013, 135, 40, 14936-14939). To a solution of succinimidyl diazoacetate (Gupta, A. K. et. al., Angew. Chem. Int. Ed. 2019, 58, 11, 3361-3367.; Ouihia, A. et. al., J. Org. Chem. 1993, 58, 1641.) (1 equiv.) in CH₂Cl₂ (0.12 M), amino acid methyl ester (1.5 equiv.) was added, and was stirred at room temperature overnight. The reaction mixture was be quenched with brine, and was extracted with CH₂Cl₂ (3×). The organic layers were then be combined, dried over sodium sulfate, filtered, and concentrated under reduced pressure to give N-Diazo-XX-OMe.

N-Diazo-Met-OMe: methyl (2-diazoacetyl)-L-methioninate was obtained with 42% yield using standard method 1 procedure for preparation of N-Diazo-XX-OMe. ¹H NMR (500 MHz, CDCl₃) δ 5.89 (br.s, 1H), 4.84 (s, 1H), 4.78 (s, 1H), 3.77 (s, 3H), 2.57-2.49 (m, 2H), 2.25-2.10 (m, 1H), 2.11 (s, 3H), 2.05-1.92 (m, 1H). 13C NMR (126 MHz, CDCl3) δ 172.9, 165.6, 52.8(+), 52.0(+), 47.6(+), 32.1(−), 30.1(−), 15.6(+).

N-Diazo-Tyr-OMe: methyl (S)-3-(4-(tert-butoxy)phenyl)-2-(2-diazoacetamido)propanoate was obtained with 60% yield using standard method 1 procedure for preparation of N-Diazo-XX-OMe. ¹H NMR (500 MHz, CDCl3) δ 7.03-6.96 (m, 2H), 6.96-6.82 (m, 2H), 5.49 (br.s, 1H), 4.90 (s, 1H), 4.74 (s, 1H), 3.71 (s, 3H), 3.16-3.02 (m, 2H), 1.34 (s, 9H). 13C NMR (126 MHz, CDCl3) δ 172.4, 165.7, 154.7, 130.6, 129.8(+), 124.4(+), 78.6, 53.6(+), 52.5(+), 47.6(+), 37.7(−), 29.0(+).

### Synthesis of N-Terminal Diazo-Building Blocks; Method 2:

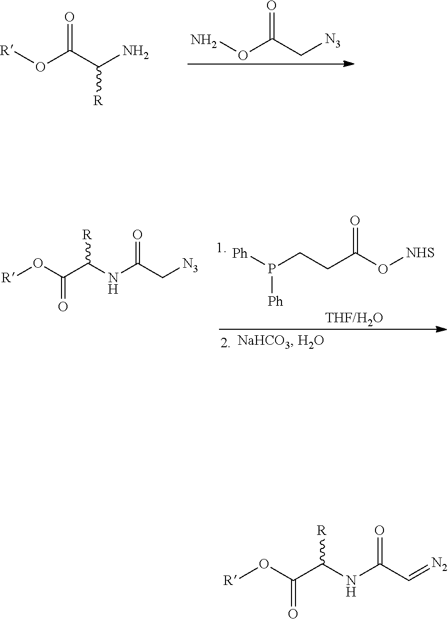

Step 1: Preparation of N-Azido-XX-OMe. N-Azido-XX-OMe was prepared following a modified procedure of Chou and Raines. (Ref: Chou, H.-H.; Raines, R. T. J. Am. Chem. Soc. 2013, 135, 40, 14936-14939.) To a solution of succinimidyl 2-azidoacetate (Ref: Vogel, K.; Glettenberg, M.; Schroeder, H.; Niemeyer, C. M. Small 2012, 9, 2, 255-262.) (1 equiv.) in CH₂Cl₂ (0.12 M), amino acid methyl ester (1.5 equiv.) was added and stirred at room temperature for overnight. The reaction mixture was que [symbol] CH₂Cl₂ (3×). The organic layers were combined, dried ov [symbol] reduced pressure to give N-Azido-XX-OMe.

Step 2: Preparation of N-Diazo-XX-OMe. Following the azide deimidogenation protocols, (Ref: Myers, E. L.; Raines, R. T. Angew. Chem. Int. Ed. 2009, 48, 13, 2359-2363.) succinimidyl 3-(diphenylphosphaneyl)propanoatephosphine (1.05 equiv.) was added to a solution of N-Azido-XX-OMe (1 equiv.) in 10% H₂O in THF (0.13 M) and stirred overnight under Ar(g). Sat. NaHCO₃(aq) was then added, and the mixture was stirred for 2 h. The mixture was then diluted with brine and extracted with CH₂Cl₂ (3×). The combined organic layers were dried over Na₂SO₄(s), filtered, and evaporated under reduced pressure. The residue was purified by silica gel flash chromatography, eluted with EtOAc/Hex, to give N-Diazo-XX-OMe.

Methyl esters (R′=Me):

N-Diazo-Gly-OMe: Step 1: Succinimidyl 2-azidoacetate (1000 mg, 5.04 mmol), and glycine methyl ester (676 mg, 7.58 mmol) were used to yield N-Azido-Gly-OMe as colorless oil (450 mg, 2.61 mmol, 52% yield). ¹H NMR (500 MHz, CDCl₃) δ 6.83 (s, 1H), 4.09 (s, 1H), 4.08 (s, 1H), 4.05 (s, 2H), 3.78 (s, 3H). ¹³C NMR (126 MHz, CDCl₃) δ 169.9, 167.0, 52.7(+), 52.6(−), 41.1(−). Step 2: Succinimidyl 3-(diphenylphosphaneyl)propanoatephosphine (1200 mg, 3.38 mmol), and N-Azido-Gly-OMe (554 mg, 3.22 mmol) were used. Purification by silica gel flash chromatography, eluted with 50% EtOAc/Hex, afforded N-Diazo-Gly-OMe as yellow oil (265 mg, 1.69 mmol, 52% yield). ¹H NMR (500 MHz, CDCl₃) δ 5.60 (s, 1H), 4.82 (br.s, 1H), 4.10 (d, J=5.2 Hz, 2H), 3.77 (s, 3H). N-Diazo-Val-OMe: Step 1: Succinimidyl 2-azidoacetate (500 g, 2.52 mmol), and valine methyl ester (497 mg, 3.79 mmol) were used to yield N-Azido-Val-OMe as colorless oil (530 mg, 2.47 mmol, 98% yield). ¹H NMR (500 MHz, CDCl₃) δ 6.83-6.68 (m, 1H), 4.56 (dd, J=8.9, 4.9 Hz, 1H), 4.14-3.94 (m, 2H), 3.76 (s, 3H), 2.20 (heptd, J=6.9, 4.8 Hz, 1H), 0.93 (dd, J=12.9, 6.9 Hz, 6H). ¹³C NMR (126 MHz, CDCl₃) δ 172.1, 166.6, 57.1(+), 52.7(−), 52.6(+), 31.4(+), 19.1(+), 17.9(+). Step 2: Succinimidyl 3-(diphenylphosphaneyl)propanoatephosphine (923 mg, 2.60 mmol), and N-Azido-Val-OMe (530 mg, 2.47 mmol) were used. Purification by silica gel flash chromatography, eluted with 50% EtOAc/Hex, afforded N-Diazo-Val-OMe as yellow oil (306 mg, 1.54 mmol, 62% yield). 1H NMR (500 MHz, Chloroform-d) δ 5.8 (d, J=8.9 Hz, 1H), 4.8 (s, 1H), 4.7-4.6 (m, 1H), 3.7 (s, 3H), 2.2 (pd, J=6.9, 4.9 Hz, 1H), 0.9 (d, J=6.9 Hz, 3H), 0.9 (d, J=6.9 Hz, 3H). ¹³C NMR (126 MHz, CDCl₃) δ 170.8, 165.7, 52.6(+), 47.6(+), 41.7(−).

N-Diazo-Pro-OMe: Step 1: Succinimidyl 2-azidoc [symbol] proline methyl ester (490 mg, 3.79 mmol) were used to y [symbol] (530 mg, 2.50 mmol, 99% yield). ¹H NMR (500 MHz, CDCl₃) δ 4.55 (dd, J=8.7, 3.6 Hz, 0.8H), 4.36 (dd, J=8.1, 2.9 Hz, 0.2H), 3.90 (d, J=1.3 Hz, 2H), 3.78-3.76 (m, 0.8H), 3.74 (s, 2.4H), 3.61-3.56 (m, 1H), 3.47-3.41 (m, 1H), 2.24-2.15 (m, 1H), 2.14-2.06 (m, 0.8H), 2.06-1.97 (m, 1.8H), 1.96-1.85 (m, 0.4H). ¹³C NMR (126 MHz, CDCl₃) δ 172.3, 166.3, 59.0(+), 52.6(+), 50.9(−), 46.4(−), 29.1(−), 24.9(−). Step 2: Succinimidyl 3-(diphenylphosphaneyl)propanoatephosphine (874 mg, 2.46 mmol), and N-Azido-Pro-OMe (530 mg, 2.34 mmol) were used. Purification by silica gel flash chromatography, eluted with 50% EtOAc/Hex, afforded N-Diazo-Pro-OMe as yellow oil (346 mg, 1.75 mmol, 75% yield).

N-Diazo-Leu-OMe: Step 1: methyl (2-azidoacetyl)-L-leucinate was obtained with 84% yield using standard procedure for preparation of N-Azido-XX-OMe. ¹H NMR (500 MHz, CDCl₃) δ 6.64 (d, J=8.5 Hz, 1H), 4.65 (td, J=8.7, 4.9 Hz, 1H), 4.03 (d, J=2.1 Hz, 2H), 3.74 (s, 3H), 1.71-1.49 (m, 3H), 0.94 (d, J=6.0 Hz, 6H). ¹³C NMR (126 MHz, CDCl₃) δ 173.1, 166.5, 52.6(+), 52.6(−), 50.7(+), 41.5(+), 25.0(+), 22.9(+), 22.0(+). Step 2: methyl (2-diazoacetyl)-L-leucinate was obtained with 63% yield using standard procedure for preparation of N-Diazo-XX-OMe. ¹H NMR (500 MHz, CDCl₃) δ 5.69 (br.s, 1H), 4.81 (s, 1H), 4.77-4.59 (m, 1H), 3.73 (s, 3H), 1.73-1.57 (m, 2H), 1.55-1.46 (m, 1H), 0.95 (d, J=6.4 Hz, 3H), 0.93 (d, J=6.6 Hz, 3H). ¹³C NMR (126 MHz, CDCl₃) δ 174.3, 165.6, 52.5(+), 51.1(+), 47.5(+), 42.0(−), 25.0(+), 22.9(+), 22.0(+).

N-Diazo-Ile-OMe: Step 1: methyl (2-azidoacetyl)-L-isoleucinate was obtained with 84% yield using standard procedure for preparation of N-Azido-XX-OMe. ¹H NMR (500 MHz, CDCl₃) δ 6.77 (d, J=8.8 Hz, 1H), 4.59 (dd, J=8.8, 4.9 Hz, 1H), 4.10-3.93 (m, 2H), 3.75 (s, 3H), 1.97-1.86 (m, 1H), 1.50-1.38 (m, 1H), 1.26-1.12 (m, 1H), 0.96-0.85 (m, 6H). Step 2: methyl (2-diazoacetyl)-L-isoleucinate was obtained with 51% yield using standard procedure for preparation of N-Diazo-XX-OMe. ¹H NMR (500 MHz, CDCl₃) δ 5.66 (d, J=8.7 Hz, 1H), 4.81 (s, 1H), 4.72-4.60 (m, 1H), 3.75-3.72 (m, 3H), 1.95-1.86 (m, 1H), 1.49-1.35 (m, 1H), 1.23-1.11 (m, 1H), 0.91 (t, J=7.2 Hz, 3H), 0.91 (d, J=7.0 Hz, 3H). ¹³C NMR (126 MHz, CDCl₃) δ 173.0, 165.5, 56.8(+), 52.3(+), 47.6(+), 38.4(+), 25.3(−), 15.6(+), 11.7(+).

N-Diazo-Phe-OMe: Step 1: methyl (2-azidoacetyl)phenylalaninate was obtained with 74% yield using standard procedure for preparation of N-Azido-XX-OMe. ¹H NMR (500 MHz, CDCl₃) δ 7.35-7.23 (m, 3H), 7.13-7.07 (m, 2H), 6.71 (d, J=8.0 Hz, 1H), 4.88 (dt, J=8.1, 5.9 Hz, 1H), 4.01-3.90 (m, 2H), 3.74 (s, 3H), 3.14 (qd, J=13.9, 5.9 Hz, 2H). ¹³C NMR (126 MHz, CDCl₃) δ 171.3, 166.2, 135.3, 129.1(+), 128.6(+), [symbol] 37.7(+). Step 2: methyl (2-diazoacetyl)-L-phenylalaninate [symbol] using standard procedure for preparation of N-Diazo-XX-OMe. ¹H NMR (500 MHz, CDCl₃) δ 7.20 (qt, J=11.1, 4.4 Hz, 3H), 7.02 (d, J=7.3 Hz, 2H), 5.65 (br.s, 1H), 4.90-4.84 (m, 1H), 4.69 (d, J=2.5 Hz, 1H), 3.66 (q, J=3.1 Hz, 3H), 3.09-2.97 (m, 2H). ¹³C NMR (126 MHz, CDCl₃) δ 172.5, 165.3, 135.9, 129.4(+), 128.8(+), 127.3(+), 53.6(+), 52.5(+), 47.5(+), 38.3(−).

### Synthesis of C-Terminal Diazo Building Blocks; Method 1:

Prepare following the procedure reported in: Gallo, R. D. C.; Campovilla, O. C. Jr.; Ahmad, A. Burtoloso, A. C. B. Frontiers in Chemistry, 2019, 7, 62.

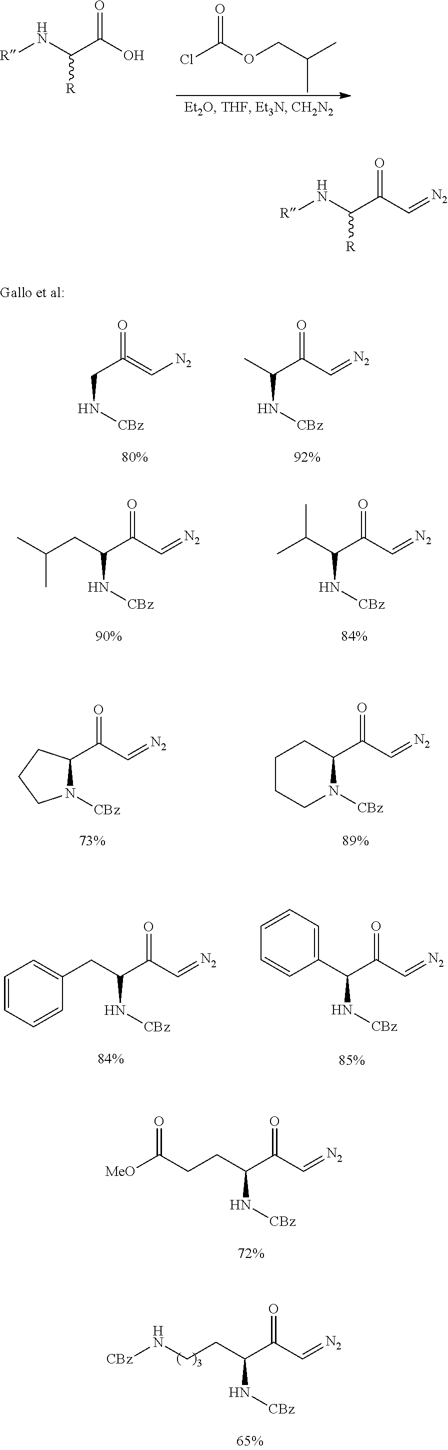

N-Fmoc-Gly-OCN2: Step 1: To a solution of (((9H-fluoren-9-yl)methoxy)carbonyl)glycine (2.5 g, 8.41 mmol) in anhydrous dichloromethane (40 mL) (COCl)₂ (1.6 g, 12.61 mmol) was added dropwise. The mixture stirred at room temperature for 2 h. The solvent was evaporated under reduced pressure, and the residue was purified by crystallization from n-hexane, to afford N-Fmoc-Gly-OCl (1.2 g, 3.9 mmol) as a white solid with 46% yield. ¹H NMR (500 MHz, CDCl₃) δ 7.77 (dd, J=7.6, 1.2 Hz, 2H), 7.58 (d, J=7.5 Hz, 2H), 7.41 (t, J=7.5 Hz, 2H), 7.32 (td, J=7.5, 1.1 Hz, 2H), 5.36 (t, J=6.3 Hz, 1H), 4.46 (d, J=6.9 Hz, 2H), 4.37 (d, J=6.2 Hz, 2H), 4.24 (t, J=6.9 Hz, 1H). ¹³C NMR (126 MHz, CDCl₃) δ 172.3, 156.0, 143.6, 141.4, 128.0, 127.3, 125.1, 120.2, 67.6, 52.4, 47.1. Step 2: To a solution of N-Fmoc-Gly-OCl (250 mg, 0.8 mmol) in 10 mL of dry DCM a 1 molar solution of (trimethylsilyl)methanimine in THF (0.871 mL) was adc [symbol] stirred at room temperature for 30 min. The solvents wer [symbol] and dry residue was fractioned on silica gel using 1:1 mixture of EtOAc and Hexanes as eluent to afford (9H-fluoren-9-yl)methyl (3-diazo-2-oxopropyl)carbamate (Rf=0.22) as a white solid (150 mg, 0.792 mmol). ¹H NMR (500 MHz, CDCl₃) δ 7.77 (d, J=7.5 Hz, 2H), 7.60 (d, J=7.5 Hz, 2H), 7.41 (t, J=7.5 Hz, 2H), 7.32 (td, J=7.5, 1.1 Hz, 2H), 5.47 (s, 1H), 5.29 (s, 1H), 4.45 (d, J=6.8 Hz, 2H), 4.22 (t, J=6.9 Hz, 1H), 3.96 (d, J=5.5 Hz, 2H).

### Synthesis of Side Chain Functionalized Diazo Building Blocks:

N-Fmoc-XX(diazo)-OR′: will be prepared following a modified procedure of Chou and Raines. (Ref: Chou, H.-H.; Raines, R. T. J. Am. Chem. Soc. 2013, 135, 40, 14936-14939.) To a solution of succinimidyl diazoacetate (Ref: Gupta, A. K.; Xiaopeng Yin, X.; Mukherjee, M.; Desai, A. A.; Mohammadlou, A.; Jurewicz, K.; Wulff, W. D. Angew. Chem. Int. Ed. 2019, 58, 11, 3361-3367.; Ouihia, A.; Rene, L.; Guilhem, J.; Pascard, C.; Badet, B. J. Org. Chem. 1993, 58, 1641.) (1 equiv.) in CH₂Cl₂ (0.12 M), N-Fmoc amino acid R′ ester (1.5 equiv.) will be added and will be stirred at room temperature for overnight. The reaction mixture will be quenched with brine and will be extracted with CH₂Cl₂ (3×). The organic layers will then be combined, dried over, filtered, and concentrated under reduced pressure to give N-Fmoc-XX(diazo)-OR′.

### Alternate Method:

### Ser/Thr (Synthesis, 2013, 45, 903):

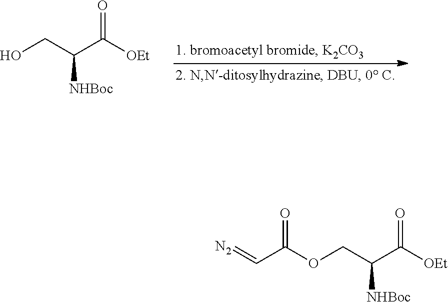

### Synthesis of N-Terminal Alkyne Building Blocks; Method 1:

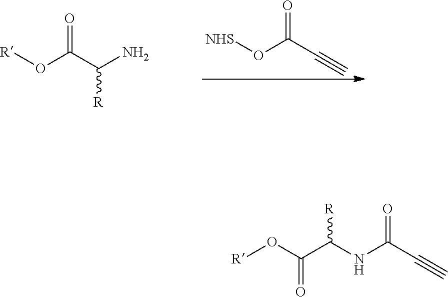

Preparation of N-Propiolyl-XX-OMe. N-Propiolyl-XX-OMe was prepared following a modified procedure of Chou and Raines (J. Am. Chem. Soc. 2013, 135, 40, 14936-14939.) To a solution of succinimidyl propiolate (Zhao, L. et. al., Adv. Funct. Mater. 2012, 22, 24, 5107-5117) (1 equiv.) in CH₂Cl₂ (0.12 M), amino acid methyl ester (1.5 equiv.) was added, and was be stirred at room temperature for overnight. The reaction mixture was quenched with brine and was extracted with CH₂Cl₂ (3×). The organic layers were [symbol] sulfate, filtered, and concentrated under reduced pressure

[symbol]

### Synthesis of N-Terminal Alkyne Building Blocks; Method 2:

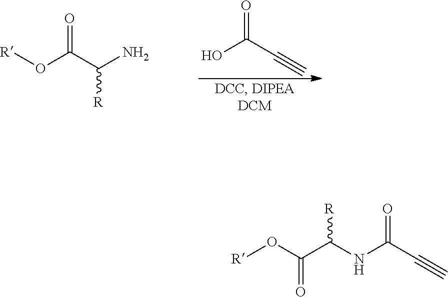

Preparation of N-propionyl-XX-OMe. Following the standard peptide coupling protocol, (Ref: Ma, W.; Bi, J.; Zhao, C.; Zhang, Z., Liu, T.; Zhang, G. Bioorg Med Chem. 2020, 28, 1, 115141) molecular sieve (3 Å) was added to a solution of propiolic acid (1 equiv.) under Ar(g) at 0° C., and stirred for 30 min. In another flask, DIPEA (0.95 equiv.) was added to a solution of amino acid methyl ester hydrochloride (1.1 equiv.) and DCC (1.05 equiv) in CH₂Cl₂ (0.8 M). This solution was added to the propiolic acid-containing mixture, stirred overnight while the ice bath was allowed to warm to room temperature. The reaction mixture was then filtered and evaporated under reduced pressure. The residue was purified by silica gel flash chromatography, eluted with 3:1 EtOAc/Hex:CH₂Cl₂, to give N-propionyl-XX-OMe.

### Synthesis of N-Terminal Alkyne Building Blocks; Method 3:

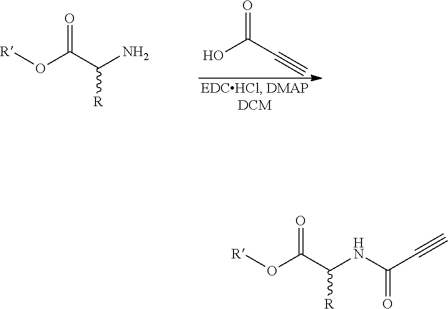

Preparation of N-Propiolyl-XX-OMe. Following the standard peptide coupling protocol, (Ref: Liu, W. R. et al. ACS Chem. Biol. 2013, 8, 8, 1664-1670.) propiolic acid (1 equiv.) was added to a solution of amino acid methyl ester hydrochloride (1.05 equiv) in dichloromethane (0.5 M), followed by dimethylaminopyridine (0.05 equiv.) and N-(3-dimethylaminopropyl)-N-ethylcarbodiimide hydrochloride (EDC, 1.2 equiv.). The reaction mixture was stirred at RT overnight. The product was extracted using DCM and washed with water (2×), brine (1×), dried over sodium sulfate, and evaporated under reduced pressure. The residue was purified by silica gel flash chromatography, eluted with 3:1 EtOAc/Hex:CH₂Cl₂, to give N-propionyl-XX-OMe.

### Methyl Esters (R′=Me):

N-Propiolyl-Val-OMe: Propiolic acid (500 mg, [symbol] mmol), valine methyl ester hydrochloride (1.3 g, 7.9 mm [symbol] used in the synthesis of N-propionyl-Val-OMe. Purification by silica gel flash chromatography, eluted with 3:1 50% EtOAc/Hex:CH₂Cl₂, gave N-propionyl-Val-OMe as yellowish oil (1.1 g, 12.4 mmol, 84% yield). ¹H NMR (500 MHz, CDCl₃) δ 6.39 (br.s, 1H), 4.61 (dd, J=8.9, 4.8 Hz, 1H), 3.76 (s, 3H), 2.85 (s, 1H), 2.25-2.15 (m, 1H), 0.96 (d, J=6.9 Hz, 3H), 0.93 (d, J=6.9 Hz, 3H). ¹³C NMR (126 MHz, CDCl₃) δ 171.7, 152.0, 76.9, 74.2, 57.5(+), 52.6(+), 31.6(+), 19.0(+), 17.9(+).

N-Propiolyl-Ala-OMe: Methyl propioloyl-L-alaninate was synthesized using standard peptide coupling protocol using EDC in 45% yield. ¹H NMR (500 MHz, CDCl₃) δ 6.51 (br.s, 1H), 4.66-4.58 (m, 1H), 3.77 (s, 3H), 2.82 (s, 1H), 1.45 (d, J=7.2 Hz, 3H). ¹³C NMR (126 MHz, CDCl₃) δ 172.7, 151.5, 77.0, 73.9, 52.9(+), 48.5(+), 18.4(+).

N-Propiolyl-Leu-OMe: Methyl propioloyl-L-leucinate was synthesized using standard peptide coupling protocol using DCC in 50% yield. ¹H NMR (500 MHz, CDCl₃) δ 6.29 (br.s, 1H), 4.68 (td, J=8.8, 4.9 Hz, 1H), 3.75 (s, 3H), 2.84 (s, 1H), 1.74-1.51 (m, 3H), 1.01-0.90 (m, 6H). ¹³C NMR (126 MHz, CDCl₃) δ 172.8, 151.8, 76.9, 74.2, 52.7(+), 51.0(+), 41.6(−), 24.9(+), 22.9(+), 22.0(+). HRMS (ESI) calcd. for C₁₀H₁₅NNaO₃ [M+Na]⁺ 220.0950, found 220.0945

N-Propiolyl-Ile-OMe: Methyl propioloyl-L-isoleucinate was synthesized using standard peptide coupling protocol using DCC in 67% yield. ¹H NMR (500 MHz, CDCl₃) δ 6.39 (br.s, 1H), 4.64 (dd, J=8.8, 4.8 Hz, 1H), 3.76 (s, 3H), 2.83 (s, 1H), 1.98-1.86 (m, 1H), 1.51-1.39 (m, 1H), 1.26-1.13 (m, 1H), 0.95 (t, 3H), 0.92 (d, J=7.0 Hz, 3H). ¹³C NMR (126 MHz, CDCl₃) δ 171.7, 151.8, 77.2, 74.1, 56.8(+), 52.5(+), 38.2(+), 25.3 (−), 15.5(+), 11.7(+).

N-Propiolyl-Phe-OMe: Methyl propioloyl-L-phenylalaninate was synthesized using standard peptide coupling protocol using EDC in 74% yield. ¹H NMR (500 MHz, CDCl₃) δ 7.35-7.23 (m, 3H), 7.15-7.06 (m, 2H), 6.36 (br.s, 1H), 4.92 (dt, J=7.8, 5.5 Hz, 1H), 3.75 (s, 3H), 3.23-3.10 (m, 2H), 2.82 (s, 1H). ¹³C NMR (126 MHz, CDCl₃) δ 171.1, 151.5, 135.3, 129.4(+), 128.8(+), 127.5(+), 76.8, 74.2, 53.6(+), 52.8(+), 37.7(−). HRMS (ESI) calcd. for C₁₃H₁₃NNaO₃ [M+Na]⁺ 254.0793, found 254.0791.

N-Propiolyl-Tyr-OMe: Methyl (S)-3-(4-(tert-butoxy)phenyl)-2-propiolamidopropanoate was synthesized using standard peptide coupling protocol using DCC in 61% yield. ¹H NMR (500 MHz, CDCl₃) δ 7.05-6.96 (m, 2H), 6.96-6.89 (m, 2H), 6.34 (br.s, 1H), 4.88 (dt, J=7.9, 5.7 Hz, 1H), 3.72 (s, 3H), 3.12-3.09 (m, 2H), 2.81 (s, 1H), 1.33 (s, 9H). ¹³C NMR (126 MHz, CDCl₃) δ 171.2, 154.8, 151.5, [symbol] 74.1, 53.7(+), 52.6(+), 37.2(−), 29.0(+).

N-Propiolyl-Asp-OMe: 4-(tert-butyl) 1-methyl propioloyl-L-aspartate was synthesized using standard peptide coupling protocol using EDC in 40% yield. ¹H NMR (500 MHz, CDCl₃) δ 6.90 (br.s, 1H), 4.83 (dt, J=8.5, 4.4 Hz, 1H), 3.78 (s, 3H), 2.96 (dd, J=17.1, 4.3 Hz, 1H), 2.85 (s, 1H), 2.77 (dd, J=17.1, 4.4 Hz, 1H), 1.44 (s, 9H). ¹³C NMR (126 MHz, CDCl₃) δ 170.6, 170.1, 151.8, 82.3, 76.9, 74.4, 53.0(+), 48.9(+), 37.2(+), 28.2(+).

N-Propiolyl-Glu-OMe: 5-(tert-butyl) 1-methyl propioloyl-L-glutamate was synthesized using standard peptide coupling protocol using DCC in 71% yield. ¹H NMR (500 MHz, CDCl₃) § 6.68 (br.s, 1H), 4.64 (td, J=7.9, 5.0 Hz, 1H), 3.77 (s, 3H), 2.83 (s, 1H), 2.41-2.23 (m, 2H), 2.23-2.12 (m, 1H), 2.06-1.89 (m, 1H), 1.45 (s, 9H). ¹³C NMR (126 MHz, CDCl₃) δ 172.2, 171.6, 151.9, 81.3, 76.9, 74.2, 52.9(+), 52.2(+), 31.4(−), 28.2(+), 27.2(−).

N-Propiolyl-Ser-OMe: methyl O-(tert-butyl)-N-propioloyl-L-serinate was synthesized using standard peptide coupling protocol using DCC in 72% yield. ¹H NMR (500 MHz, CDCl₃) δ 6.71 (br.s, 1H), 4.74 (dt, J=8.5, 2.9 Hz, 1H), 3.82 (dd, J=9.2, 2.8 Hz, 1H), 3.76 (s, 3H), 3.59 (dd, J=9.2, 3.1 Hz, 1H), 2.85 (s, 1H), 1.14 (s, 9H). ¹³C NMR (126 MHz, CDCl₃) δ 169.9, 151.6, 76.9, 73.9, 73.6, 61.5(−), 53.0(+), 52.5(+), 27.1(+).

N-Propiolyl-Thr-OMe: methyl O-(tert-butyl)-N-propioloyl-L-threoninate was synthesized using standard peptide coupling protocol using DCC in 64% yield. ¹H NMR (500 MHz, CDCl₃) δ 6.61 (br.s, 1H), 4.55 (dd, J=9.4, 1.8 Hz, 1H), 4.24 (qd, J=6.3, 1.8 Hz, 1H), 3.73 (s, 3H), 2.87 (s, 1H), 1.20 (d, J=6.3 Hz, 3H), 1.12 (s, 9H). ¹³C NMR (126 MHz, CDCl₃) δ 170.5, 152.6, 76.9, 74.5, 74.4, 67.4(+), 58.1(+), 52.5(+), 28.5(+), 21.2(+).

N-Propiolyl-Lys-OMe: methyl N⁶-((benzyloxy)carbonyl)-N²-propioloyl-L-lysinate was synthesized using standard peptide coupling protocol using DCC in 87% yield. ¹H NMR (500 MHz, CDCl₃) δ 7.39-7.30 (m, 5H), 6.66 (br.s, 1H), 5.10 (s, 2H), 4.83 (br.s, 1H), 4.61 (td, J=7.8, 4.9 Hz, 1H), 3.75 (s, 3H), 3.26-3.10 (m, 2H), 2.82 (s, 1H), 1.89 (m, 1H), 1.75 (m, 2H), 1.59-1.45 (m, 2H), 1.45-1.29 (m, 1H). ¹³C NMR (126 MHz, CDCl₃) δ 172.0, 156.8, 151.9, 136.6, 128.7(+), 128.3(+), 77.0, 74.1, 66.9(−), 52.8(+), 52.5(+), 40.4(−), 31.7(−), 29.6(−), 22.2(−).

### Synthesis of N-Terminal Alkene Building Blocks; Method 1:

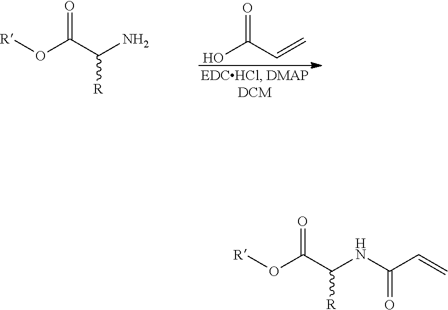

Preparation of N-Acryl-XX-OR′: N-Acryl-XX-OM [symbol] procedure of Liu and colleagues. (Ref: Liu, W. R. et al. ACS (Acrylic [symbol] acid (1 equiv.) was added to a solution of amino acid [symbol] dichloromethane (0.5 M), followed by dimethylaminopyridine (0.05 equiv.) and N-(3-dimethylaminopropyl)-N-ethylcarbodiimide hydrochloride (EDC, 1.2 equiv.). The reaction mixture was stirred at RT overnight. The product was extracted by DCM and washed with water (2×), brine (1×), dried over sodium sulfate, and evaporated under reduced pressure. The residue was purified by silica gel flash chromatography, eluted with 3:1 EtOAc/Hex:CH₂Cl₂, to give N-Acryl-XX-OMe.

N-Acryl-Ala-OMe: methyl acryloyl-L-alaninate was obtained with 43% yield using method 1 for preparation of N-Acryl-XX-OR′. ¹H NMR (500 MHz, CDCl₃) δ 6.31 (dd, J=17.0, 1.4 Hz, 1H), 6.17 (br.s, 1H), 6.13 (dd, J=17.0, 10.3 Hz, 1H), 5.68 (dd, J=10.3, 1.4 Hz, 1H), 4.69 (q, J=7.2 Hz, 1H), 3.77 (s, 3H), 1.45 (d, J=7.1 Hz, 3H). ¹³C NMR (126 MHz, CDCl₃) δ 173.7, 164.9, 130.5(+), 127.3(−), 52.7(+), 48.2(+), 18.7(+).

N-Acryl-Val-OMe: methyl acryloyl-L-valinate was obtained with 50% yield using method 1 for preparation of N-Acryl-XX-OR′. ¹H NMR (500 MHz, CDCl₃) δ 6.32 (dd, J=17.0, 1.4 Hz, 1H), 6.16 (dd, J=17.0, 10.2 Hz, 1H), 6.10 (br.s, 1H), 5.69 (dd, J=10.2, 1.4 Hz, 1H), 4.67 (dd, J=8.8, 4.9 Hz, 1H), 3.75 (s, 3H), 2.20 (dtd, J=13.8, 6.9, 4.2 Hz, 1H), 0.95 (d, J=6.9 Hz, 3H), 0.92 (d, J=6.9 Hz, 3H). ¹³C NMR (126 MHz, CDCl₃) δ 172.7, 165.4, 130.5(+), 127.5(−), 57.1(+), 52.4(+), 31.6(+), 19.1(+), 18.0(+). HRMS: m/z calculated for C₉H₁₅NNaO₃ [M+Na], 208.095; found 208.0945.

N-Acryl-Leu-OMe: methyl acryloyl-L-leucinate was obtained with 79% yield using method 1 for preparation of N-Acryl-XX-OR′. ¹H NMR (500 MHz, CDCl₃) δ 6.32 (dd, J=17.0, 1.3 Hz, 1H), 6.13 (dd, J=16.9, 10.3 Hz, 1H), 5.97 (br.s, 1H), 5.69 (dd, J=10.2, 1.4 Hz, 1H), 4.75 (td, J=8.7, 4.9 Hz, 1H), 3.75 (s, 3H), 1.73-1.65 (m, 2H), 1.61-1.52 (m, 1H), 1.00-0.89 (m, 6H). ¹³C NMR (126 MHz, CDCl₃) δ 173.7, 165.2, 130.3(+), 127.6(−), 52.6(+), 50.8(+), 42.0(−), 25.0(+), 22.9(+), 22.1(+). HRMS: m/z calculated for C₁₀H₁₇NNa₀₃ [M+Na], 222.1106; found 222.1102

N-Acryl-Ile-OMe: methyl propioloyl-L-isoleucinate was obtained with 56% yield using method 1 for preparation of N-Acryl-XX-OR′. ¹H NMR (500 MHz, CDCl₃) δ 6.32 (dd, J=16.9, 1.4 Hz, 1H), 6.15 (dd, J=17.0, 10.2 Hz, 1H), 6.09 (br.s, 1H), 5.69 (dd, J=10.2, 1.4 Hz, 1H), 4.70 (dd, J=8.6, 4.9 Hz, 1H), 3.75 (s, 3H), 1.92 (dqt, J=9.4, 6.9, 4.8 Hz, 1H), 1.45 (dtd, J=14.8, 7.4, 4.6 Hz, 1H), 1.29-1.13 (m, 1H), 0.96-0.89 (m, 6H). ¹³C NMR (126 MHz, CDCl₃) δ 172.7, 165.2, 130.5(+), 127.5(−), 56.5(+), 52.4(+), 38.3(+), 25.4(−), 15.5(+), 11.7(+). HRMS: m/z calculated for C₁₀H₁₇NNaO₃ [M+Na], 222.1106; found 222.1100.

N-Acryl-Pro-OMe: methyl acryloyl-L-prolinate [symbol] method 1 for preparation of N-Acryl-XX-OR′. ¹H NMR [symbol] 2H), 5.77-5.61 (m, 1H), 4.56 (dd, J=8.3, 3.9 Hz, 1H), 3.75 (s, 3H), 3.71-3.55 (m, 2H), 2.33-2.08 (m, 1H), 2.06-1.83 (m, 3H). ¹³C NMR (126 MHz, CDCl₃) δ 172.8 (d, J=6.3 Hz), 164.6, 128.8(−), 128.1(+), 59.0(+), 52.5(+), 47.1(−), 29.3(−), 25.0(−). HRMS: m/z calculated for C9H13NNaO3 [M+Na], 206.0793; found 206.0791.

N-Acryl-Phe-OMe: methyl acryloyl-L-phenylalaninate was obtained with 49% yield using method 1 for preparation of N-Acryl-XX-OR′. ¹H NMR (500 MHz, CDCl₃) δ 7.27 (d, J=14.4 Hz, 3H), 7.11-7.05 (m, 2H), 6.30 (dd, J=17.0, 1.3 Hz, 1H), 6.09 (dd, J=17.0, 10.3 Hz, 1H), 6.00 (br.s, 1H), 5.68 (dd, J=10.3, 1.3 Hz, 1H), 4.98 (dt, J=7.8, 5.5 Hz, 1H), 3.75 (s, 3H), 3.24-3.12 (m, 2H). ¹³C NMR (126 MHz, CDCl₃) δ 172.0, 165.0, 135.8, 130.3(+), 129.4(+), 128.7(+), 127.6(−), 127.3(+), 53.2(+), 52.6(+), 37.9(−). HRMS: m/z calculated for C13H15NNaO3 [M+Na], 256.095; found 256.0948

N-Acryl-Tyr-OMe: methyl (S)-2-acrylamido-3-(4-(tert-butoxy)phenyl)propanoate was obtained with 45% yield using method 1 for preparation of N-Acryl-XX-OR′. ¹H NMR (500 MHz, CDCl₃) δ 7.00-6.94 (m, 2H), 6.93-6.86 (m, 2H), 6.29 (dt, J=17.0, 1.4 Hz, 1H), 6.09 (dd, J=17.0, 10.3 Hz, 1H), 6.05 (br.s, 1H), 5.67 (dt, J=10.3, 1.6 Hz, 1H), 4.97-4.90 (m, 1H), 3.71 (d, J=1.6 Hz, 3H), 3.12 (dd, J=5.8, 2.5 Hz, 2H), 1.32 (s, 9H). ¹³C NMR (126 MHz, CDCl₃) δ 172.1, 164.9, 154.6, 130.6, 130.3(+), 129.8(+), 127.5(−), 124.4(+), 78.6, 53.3(+), 52.5(+), 37.3(−), 28.9(+). HRMS: m/z calculated for C17H23NNaO4 [M+Na], 328.1525; found 328.1523

N-Acryl-Asp-OMe: 4-(tert-butyl) 1-methyl acryloyl-L-aspartate was obtained with 40% yield using method 1 for preparation of N-Acryl-XX-OR′. ¹H NMR (500 MHz, CDCl₃) δ 6.59 (br.s, 1H), 6.32 (dd, J=17.0, 1.4 Hz, 1H), 6.15 (dd, J=17.0, 10.3 Hz, 1H), 5.70 (dd, J=10.3, 1.4 Hz, 1H), 4.90 (dt, J=8.4, 4.4 Hz, 1H), 3.77 (s, 3H), 2.97 (dd, J=17.0, 4.4 Hz, 1H), 2.79 (dd, J=17.0, 4.5 Hz, 1H), 1.43 (s, 9H). ¹³C NMR (126 MHz, CDCl₃) δ 171.5, 170.4, 165.1, 130.5(+), 127.5(−), 82.0, 52.9(+), 48.8(+), 37.6(−), 28.2(+)

N-Acryl-Glu-OMe: 5-(tert-butyl) 1-methyl acryloyl-L-glutamate was obtained with 58% yield using method 1 for preparation of N-Acryl-XX-OR′. ¹H NMR (500 MHz, CDCl₃) δ 6.45 (br.s, 1H), 6.31 (dd, J=17.0, 1.3 Hz, 1H), 6.13 (dd, J=17.0, 10.3 Hz, 1H), 5.69 (dd, J=10.3, 1.3 Hz, 1H), 4.67 (td, J=7.9, 4.9 Hz, 1H), 3.75 (s, 3H), 2.42-2.23 (m, 2H), 2.17 (dtd, J=14.3, 7.2, 4.9 Hz, 1H), 2.06-1.94 (m, 1H), 1.43 (s, 9H). ¹³C NMR (126 MHz, CDCl₃) δ 172.5, 165.4, 130.4(+), 127.5(−), 81.1, 52.7(+), 52.0(+), 31.6(−), [symbol] calculated for C₁₃H₂₁NNaO₅ [M+Na], 294.1317; found

[symbol]

N-Acryl-Ser-OMe: methyl N-acryloyl-O-(tert-butyl)-L-serinate was obtained with 63% yield using method 1 for preparation of N-Acryl-XX-OR′. ¹H NMR (300 MHz, CDCl₃) δ 6.41 (br.s, 1H), 6.37-6.27 (m, 1H), 6.25-6.12 (m, 1H), 5.69 (dd, J=10.0, 1.3 Hz, 1H), 4.80 (dd, J=8.2, 3.4 Hz, 1H), 3.84 (ddd, J=9.1, 2.8, 1.1 Hz, 1H), 3.75 (d, J=1.1 Hz, 3H), 3.60 (ddd, J=9.1, 3.1, 1.1 Hz, 1H), 1.13 (s, 9H). ¹³C NMR (126 MHz, CDCl₃) δ 171.1, 165.2, 130.5(+), 127.4(−), 73.6, 62.1(−), 53.0(+), 52.6(+), 27.4(+). HRMS: m/z calculated for C11H19NNaO4 [M+Na], 252.1212; found 252.1206.

N-Acryl-Thr-OMe: methyl N-acryloyl-O-(tert-butyl)-L-threoninate was obtained with 70% yield using method 1 for preparation of N-Acryl-XX-OR′. ¹H NMR (300 MHz, CDCl₃) δ 6.37 (br.s, 1H), 6.34-6.17 (m, 2H), 5.70 (dt, J=9.8, 1.8 Hz, 1H), 4.63 (d, J=1.7 Hz, 1H), 4.32-4.20 (m, 1H), 3.72 (d, J=1.7 Hz, 3H), 1.19 (d, J=1.7 Hz, 3H), 1.12 (s, 9H). ¹³C NMR (126 MHz, CDCl₃) δ 171.4, 165.9, 130.5(+), 127.4(−), 74.3, 67.6(+), 57.8(+), 52.4(+), 28.4(+), 21.1(+). HRMS: m/z calculated for C₁₂H₂₁NNaO₄ [M+Na], 266.1368; found 266.1367.

N-Acryl-Lys-OMe: methyl N2-acryloyl-N6-((benzyloxy)carbonyl)-L-lysinate was obtained with 62% yield using method 1 for preparation of N-Acryl-XX-OR′. ¹H NMR (500 MHz, CDCl₃) δ 7.39-7.29 (m, 5H), 6.35 (br.s, 1H), 6.31 (dd, J=17.0, 1.5 Hz, 1H), 6.15 (dd, J=17.0, 10.2 Hz, 1H), 5.66 (dd, J=10.2, 1.5 Hz, 1H), 5.08 (s, 2H), 4.90 (br.s, 1H), 4.66 (td, J=7.8, 4.8 Hz, 1H), 3.74 (s, 3H), 3.18 (q, J=6.9 Hz, 2H), 1.94-1.82 (m, 1H), 1.79-1.68 (m, 2H), 1.60-1.45 (m, 2H), 1.45-1.27 (m, 1H). ¹³C NMR (126 MHz, CDCl₃) δ 173.0, 165.4, 156.7, 136.6, 130.3(+), 128.7(+), 128.3(+), 128.2(+), 127.6(−), 66.8(−), 52.7(+), 52.0(+), 40.4(−), 32.0(−), 29.5(−), 22.2(−). HRMS: m/z calculated for C₁₈H₂₄N₂NaO₅ [M+Na], 371.1583; found 371.1587.

N-Acryl-His-OMe: methyl N2-acryloyl-N6-trityl-L-histidinate was obtained with 37% yield using method 1 for preparation of N-Acryl-XX-OR′. ¹H NMR (500 MHz, CDCl₃) δ 7.51 (d, J=7.8 Hz, 1H), 7.41-7.37 (m, 1H), 7.36-7.30 (m, 10H), 7.13-7.06 (m, 5H), 6.55 (s, 1H), 6.29 (dt, J=17.1, 1.3 Hz, 1H), 6.17 (ddd, J=17.0, 10.2, 1.2 Hz, 1H), 5.65 (dt, J=10.2, 1.3 Hz, 1H), 4.91-4.84 (m, 1H), 3.61 (d, J=1.3 Hz, 3H), 3.14-2.99 (m, 2H). ¹³C NMR (126 MHz, CDCl₃) δ 171.8, 165.4, 142.2, 138.8(+), 136.5, 130.9(+), 129.9(+), 128.3(+), 128.2(+), 126.8(−), 119.9(+), 75.5, 52.7(+), 52.4(+), 29.7(−). HRMS: m/z calculated for C₂₉H₂₇N₃NaO₃ [M+Na], 488.1950; found 488.1950

Synthesis of N-Terminal Alkene Building Blocks; Metl

[symbol]

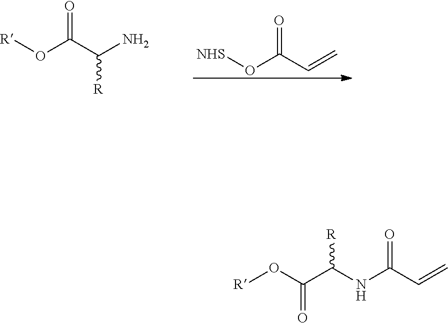

Preparation of N-Acryl-XX-OR′: N-Acryl-XX-OMe will be prepared following a modified procedure of Chou and Raines. (Ref: Chou, H.-H.; Raines, R. T. J. Am. Chem. Soc. 2013, 135, 40, 14936-14939.) To a solution of commercially available succinimidyl acrylate in CH₂Cl₂ (0.12 M), amino acid methyl ester (1.5 equiv.) will be added, and will be stirred at room temperature for overnight. The reaction mixture will be quenched with brine, and will be extracted with CH₂Cl₂ (3×). The organic layers will then be combined, dried over, filtered, and concentrated under reduced pressure to give N-Acryl-XX-OMe.

### Synthesis of C-Terminal Alkyne/Alkene Building Blocks; Method 1:

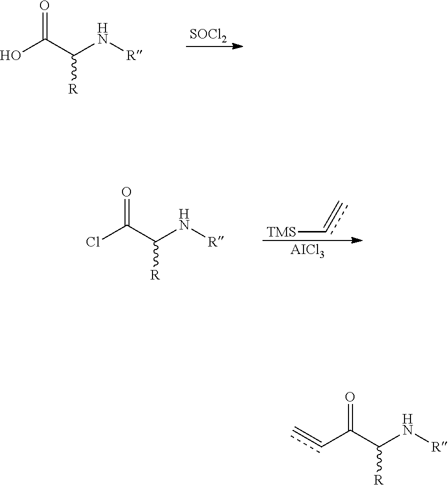

Step 1: Preparation of amino acid acyl chloride. Amino acid acyl chlorides will be prepared following reported protocol. (Ref: Carpino, L. A.; Cohen, B. J.; Stephens, K. E., Jr.; Sadat-Aalaee, Y.; Tien, J. H.; Langridge, D. C. J. Org. Chem. 1986, 51, 3732e3734.)

Step 2: Preparation of N—R″-XX-Ethyne. N—R″-XX-Ethyne will be prepared following the procedure of Wilbur and Bonner (Ref: Wilbur, J. M. Jr.; Bonner, B. A. J. Polym. Sci. A Polym. Chem. 1990, 28, 13, 3747-3759.) with some modifications. Solution of amino acid acyl chloride (1 equiv.), BTMSA (1 equiv.), and AlCl₃ (1 equiv) in CH₂Cl₂ (0.5 M) will be used. The same purification procedures will be employed.

### Synthesis of Side Chain Functionalized Alkene/Alkyne Building Blocks:

N-Fmoc-XX(yne)-OR′: will be prepared following a modified procedure of Chou and Raines. (Ref: Chou, H.-H.; Raines, R. T. J. Am. Chem. Soc. 2013, 135, 40, 14936-14939.) To a solution of succinimidyl propiolate (Ref: Zhao, L.; Chano, T.; Morikawa, S.; Saito, Y.; Shiino, A.; Shimizu, S.; Maeda, T.; Irie, T.; Aonuma, S.; Okabe, H.; Kimura, T.; Inubushi, T.; Komatsu, N. Adv. Funct. Mater. 2012, 22, 24, 5107-5117) (1 equiv.) in CH₂Cl₂ (0.12 M), the N-Fmoc amino acid R′ester (1.5 equiv.) will be added, and will be stirred at room temperature for overnight. The reaction mixture will be quenched with brine, and will be extracted with CH₂Cl₂ (3×). The organic layers will then be combined, dried ove [symbol] reduced pressure to give N-Fmoc-XX(yne)-OR′.

[symbol]

N-Fmoc-XX(ene)-OR′: will be prepared following a modified procedure of Chou and Raines. (Ref: Chou, H.-H.; Raines, R. T. J. Am. Chem. Soc. 2013, 135, 40, 14936-14939.) To a solution of commercially available succinimidyl acrylate in CH₂Cl₂ (0.12 M), the N-Fmoc amino acid R′ester (1.5 equiv.) will be added, and will be stirred at room temperature for overnight. The reaction mixture will be quenched with brine, and will be extracted with CH₂Cl₂ (3×). The organic layers will then be combined, dried over, filtered, and concentrated under reduced pressure to give N-Fmoc-XX(ene)-OR′.

The complete disclosures of the patents, patent documents, and publications cited herein are incorporated by reference in their entirety as if each were individually incorporated. To the extent that there is any conflict or discrepancy between this specification as written and the disclosure in any document that is incorporated by reference herein, this specification as written will control. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows.

1. A pyrazole-containing peptide of one of formulas, 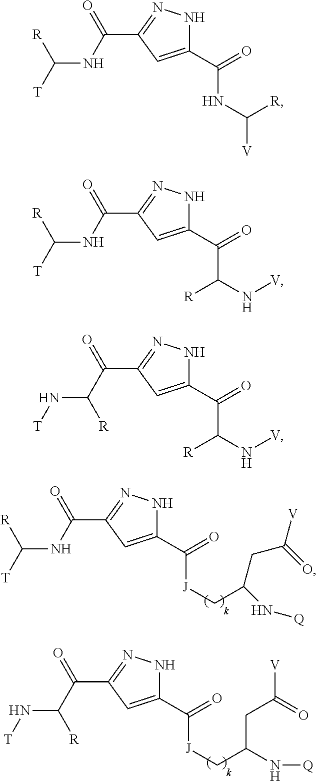
or an isomer thereof,
wherein:
each R is independently an amino acid side chain;
T, V, and Q are each independently an amino acid, a peptide, C(O)OH, H, an amine protecting group or a carboxylic acid protecting group;
k is 0, 1, 2, 3, 4, 5, 6, 7, or 8; and
J is one of the formulas 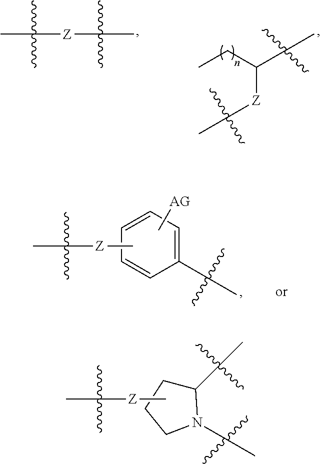
wherein:
each Z is independently O, N, NH, S, SH, or Se;
n is 0, 1, 2, or 3; and
AG is a halogen or alkyl.

2. A pyrazoline-containing peptide of one of formulas, 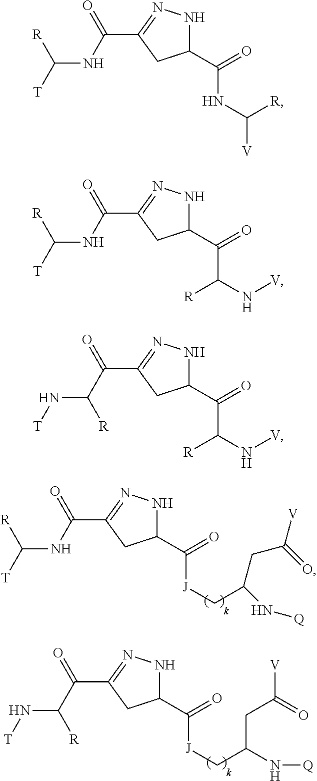
or an isomer thereof,
wherein:
each R is independently an amino acid side chain;
T, V, and Q are each independently an amino acid, a peptide, C(O)OH, H, an amine protecting group or a carboxylic acid protecting group,
k is 0, 1, 2, 3, 4, 5, 6, 7, or 8; and
J is one of the formulas 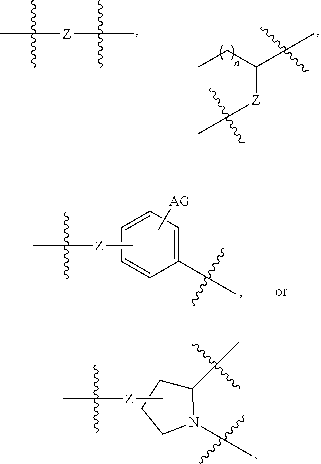
wherein:
each Z is independently O, N, NH, S, SH, or Se;
n is 0, 1, 2, or 3; and
AG is a halogen or alkyl.

3. The pyrazole-containing peptide or pyrazoline-containing peptide of claim 1, wherein at least one of T, Q, or V is a peptide.

4. The pyrazole-containing peptide or pyrazoline-containing peptide of claim 2, wherein at least one wherein T, Q, or V is a peptide.

5. (canceled)

6. (canceled)

7. The pyrazole-containing peptide or pyrazoline-containing peptide of claim 1, wherein;
T and B are covalently coupled through a cyclic peptide;
T and Q are covalently coupled through a cyclic peptide; or
V and Q are covalently coupled through a cyclic peptide.

8. The pyrazole-containing peptide or pyrazoline-containing peptide of claim 2, wherein:
T and B are covalently coupled through a cyclic peptide;
T and Q are covalently coupled through a cyclic peptide; or
V and Q are covalently coupled through a cyclic peptide.

9. (canceled)

10. The pyrazole-containing peptide or pyrazoline-containing peptide of claim 1, wherein at least one of T, V, or Q is an amino acid.

11. The pyrazole-containing peptide or pyrazoline-containing peptide of claim 2, wherein at least one of T, V, or Q is an amino acid.

12. The pyrazole-containing peptide or pyrazoline-containing peptide of claim 1, wherein at least one of T, V, or Q is H or C(O)OH.

13. The pyrazole-containing peptide or pyrazoline-containing peptide of claim 2, wherein at least one of T, V, or Q is H or C(O)OH.

14-17. (canceled)

18. The pyrazole-containing peptide or pyrazoline-containing peptide of claim 1, wherein each R is independently the side chain of a proteogenic amino acid.

19. The pyrazole-containing peptide or pyrazoline-containing peptide of claim 2, wherein each R is independently the side chain of a proteogenic amino acid.

20. A method for synthesizing a pyrazole-containing peptide or a pyrazoline-containing peptide comprising:
reacting an alkyne functionalized amino acid of formula 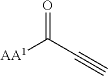
or a alkene functionalized amino acid of formula 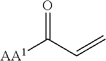
with a diazo functioanlized amino acid of formula 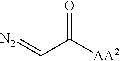
under a set of reaction conditions,
wherein AA¹ and AA² are each independently one of formulas: 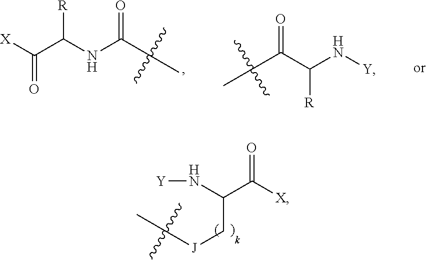
wherein:
each R is independently an amino acid side chain;
X and Y are each independently C(O)OH, H, an amine protecting group or a carboxylic acid protecting group;
k is 0, 1, 2, 3, or 4; and
J is of the of the general formula: 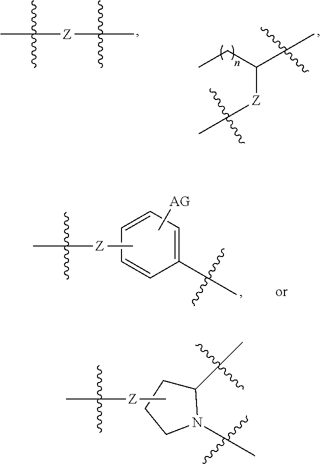
each Z is independently O, N, NH, S, SH, or Se;
n is 0, 1, 2, 3, or 4; and
AG is a halogen or alkyl.

21-35. (canceled)

36. A method of making a library of pyrazole-containing peptides comprising repeating the method of claim 20 to produce a plurality of pyrazole-containing peptides.

37. A method of making a library of pyrazoline-containing peptides comprising repeating the method of claim 20 to produce a plurality of pyrazoline-containing peptides.

38. A method of making a library of synthetic amino acid-derived compounds, the method comprising:
providing building blocks comprising a set of diazo-functional amino acids and a set of alkene- or alkyne-functional amino acids; and
reacting one of the set of diazo-functional amino acids with one of the set of alkene- or alkyne-functional amino acids under conditions effective to form a pyrazole- or pyrazoline-containing peptide cycloadduct of the library; and
repeating the reacting step with a different diazo-functional amino acid and a different alkene- or alkyne-functional amino acid under conditions effective to form a different pyrazole- or pyrazoline-containing peptide cycloadduct of the library.

39. The method of claim 38, wherein the diazo-functional amino acid is an N-terminal diazo-functional amino acid (i.e., an N-terminal amino acid-based diazo compound or a diazoacetamide), a C-terminal diazo-functional amino acid (C-terminal amino acid-based diazo compound), or a side chain diazo-functional amino acid.

40. The method of claim 38, wherein the alkene- or alkyne-functional amino acid is an N-terminal alkene- or alkyne-functional amino acid (i.e., an N-terminal amino acid-based alkene or alkyne compound), a (C-terminal alkene- or alkyne-functional amino acid (C-terminal amino acid-based alkene or alkyne compound), or a side chain alkene or alkyne-functional amino acid.

41. The method of claim 39, wherein the alkene- or alkyne-functional amino acid is an N-terminal alkene- or alkyne-functional amino acid (i.e., an N-terminal amino acid-based alkene or alkyne compound), a C-terminal alkene- or alkyne-functional amino acid (C-terminal amino acid-based alkene or alkyne compound), or a side chain alkene or alkyne-functional amino acid.

42-47. (canceled)

48. A library of synthetic amino acid-derived compounds formed by the method of claim 38.
